(19) United States
(12) Patent Application Publication
(10) Pub. No.: US 2008/0114056 A1
(43) Pub. Date: May 15, 2008

(54) Chromone Derivatives Useful as Vanilloid Antagonists

(76) Inventors: Timothy John Ritchie, West Sussex (GB); Andrew James Culshaw, West Sussex (GB); Terance William Hart, High Peak (GB); Christopher Thomas Brain, West Sussex (GB); Edward Karol Dziadulewicz, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/570,049

(22) PCT Filed: Jun. 8, 2005
(86) PCT No.: PCT/EP05/06252

§ 371 Date: Mar. 28, 2007

(30) Foreign Application Priority Data
Jun. 8, 2004 (GB) 0412768.4

(51) Int. Cl. A61K 31/352; A61P 25/00; C07D 311/28

(52) U.S. Cl. 514/456; 549/403

(57) ABSTRACT

The present invention relates to the use of a chromone compound of the formula

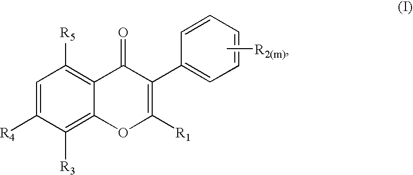

wherein
R₁, R₂, R₃, R₄, R₅ and m are as defined in the specification and in the claims, in free form or in salt form, and, where possible, in acid addition salt form, as a vanilloid antagonist.

The present invention relates to the use of chromone derivatives as vanilloid antagonists, to certain novel chromone derivatives, to processes for preparing them, to their use as pharmaceuticals and to pharmaceutical compositions containing them.

In a first aspect, the present invention relates to the use of a chromone compound of the formula

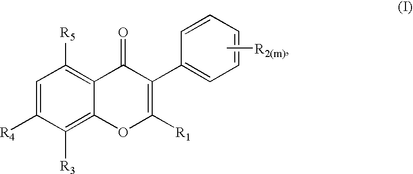

wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group

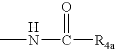

or a group

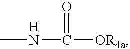

where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl;
R₅ is hydrogen, C₁-C₆alkoxy or hydroxy; and
m is 1, 2 or 3,
   in free form or in salt form, and, where possible, in acid addition salt form, as a vanilloid antagonist.

In a special embodiment of the first aspect, the present invention relates to the use of a chromone compound of the formula I, wherein
R₁ is C₁-C₆alkyl, (C₁-C₆alkyl)C₁-C₆alkyl, di-(C₁-C₆alkyl)C₁-C₆alkyl, C₃-C₆cycloalkyl or trifluoromethyl;
each R₂, independently, is halo, tri-halo substituted C₁-C₆alkyl, hydroxyC₁-C₆alkyl or a group

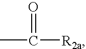

where R₂ₐ is C₁-C₆alkyl;
R₃ is hydrogen, C₁-C₆alkyl, hydroxy, C₁-C₆alkoxy or (C₃-C₆cycloalkyl)C₁-C₆alkoxy;
R₄ is hydroxy, esterified hydroxy, etherified hydroxy, amino, (C₁-C₆alkyl)amino, a group

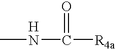

or a group

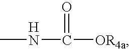

where R₄ₐ is C₁-C₆alkyl;
R₅ is hydrogen or hydroxy; and
m is 1 or 2,
   in free or salt form and, where possible, in acid addition salt form, as a vanilloid antagonist.

In a second aspect, the present invention relates to novel chromone compounds of the formula

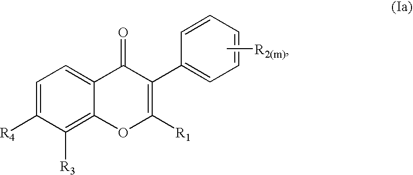

wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group

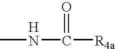

or a group

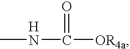

where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl; and
m is 1, 2 or 3,
   in free form or in salt form, and, where possible, in acid addition salt form, with the proviso, that, when R₂ is halo, m is 1, R₃ is hydrogen or hydroxy and R₄ is hydroxy, then R₁ is other than methyl.

In a special embodiment of the second aspect, the present invention relates to novel chromone compounds of the formula Ia, wherein
R₁ is C₁-C₆alkyl, (C₁-C₆alkyl)C₁-C₆alkyl, di-(C₁-C₆alkyl)C₁-C₆alkyl or C₃-C₆cycloalkyl;
each R₂, independently, is halo, tri-halo substituted C₁-C₆alkyl, hydroxyC₁-C₆alkyl or a group

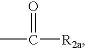

where R₂ₐ is C₁-C₆alkyl;
R₃ is hydrogen, C₁-C₆alkyl, hydroxy, C₁-C₆alkoxy or (C₃-C₆cycloalkyl)C₁-C₆alkoxy;
R₄ is hydroxy, esterified hydroxy, etherified hydroxy, amino, (C₁-C₆alkyl)amino, a group

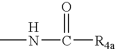

or a group

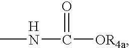

where R₄ₐ is C₁-C₆alkyl; and
m is 1 or 2,
   in free or salt form and, where possible, in acid addition salt form, with the proviso that when R₂ is halo, m is 1, R₃ is hydrogen or hydroxy and R₄ is hydroxy, then R₁ is other than methyl.

Terms used in this specification have the following meanings:

“C₁-C₆alkyl” denotes straight-chain or branched C₁ to C₆-alkyl, e.g., methyl ethyl, n-propyl, isopropyl, n-butyl, isobutyl, sec-butyl or tert-butyl.

“C₁-C₆alkoxy” denotes straight-chain or branched C₁ to C₆-alkyl-oxy, e.g., methoxy, ethoxy, n-propoxy or isopropoxy.

“Halo” or “halogen” may be 1, Br, Cl or F.

“Esterified hydroxy” denotes acyloxy, preferably C₁-C₆alkanoyloxy, more preferably C₁-C₄alkanoyloxy, or C₁-C₆alkoxycarbonyloxy.

“Etherified hydroxy” denotes C₁-C₆alkoxy, preferably C₁-C₄alkoxy, benzyloxy, —O—P(═O)(OH)₂, (C₁-C₆alkyl)pyrrolidinyloxy, a pyrazolyl-substituted C₁-C₆alkoxy group, or a 1,4-diazacyclohexyl-substituted C₁-C₆alkoxy group the heterocyclic ring of which is substituted by C₁-C₆alkyl and C₁-C₆alkoxycarbonyl.

The chromone compounds of the invention exist in free or salt form and, where possible, in acid addition salt form. The invention is to be understood as including the compounds of formulae (I) and (Ia) in free or salt form and, where possible, in acid addition salt form. In the latter connection, suitable pharmaceutically acceptable acid addition salts for pharmaceutical use in accordance with the invention include, in particular, the hydrochloride salt.

In formulae (I) and (Ia), the following significances are preferred independently, collectively or in any combination or sub-combination:
(a) R₁ is C₁-C₄alkyl, (C₁-C₄alkyl)C₁-C₄alkyl or di-(C₁-C₄alkyl)C₁-C₄alkyl;
(b) each R₂, independently, is chloro, fluoro, trifluoro-substituted C₁-C₄alkyl, more preferably trifluoromethyl, C₁-C₄alkylcarbonyl, more preferably methyl carbonyl, or hydroxyC₁-C₄alkyl, more preferably hydroxymethyl;
(c) R₃ is hydrogen, C₁-C₄alkyl, hydroxy, C₁-C₄alkoxy or (C₃-C₆cycloalkyl)C₁-C₄alkoxy; and
(d) R₄ is hydroxy, amino or (C₁-C₄alkyl)amino.

In a third aspect, the present invention relates to processes for preparing the compounds of formula (Ia) as depicted in the following reaction schemes:

### A. For Preparing Compounds of Formula (Ia), where R₁ is as Defined Above, R₂ is Chloro, R₃ is Hydrogen, R₄ is Hydroxy and m is 1.

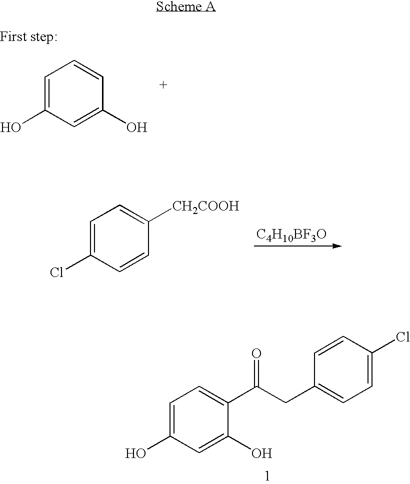

### General Description:

The first step of Scheme A involves the Friedel-Crafts acylation of resorcinol with 4-chlorophenylacetic acid in the presence of boron trifluoride etherate to obtain the ethanone compound of formula 1.

First Part of Second Step:

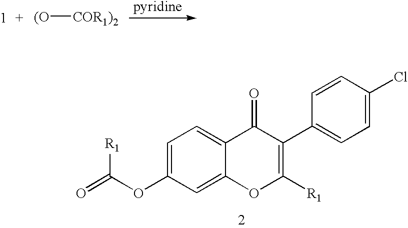

General Description:

The first part of the second step of Scheme A involves the cyclisation/esterification of the ethanone compound of formula 1 with a suitable anhydride in the presence of an organic base, e.g., pyridine, to obtain an ester compound of formula 2.

### Second Part of Second Step:

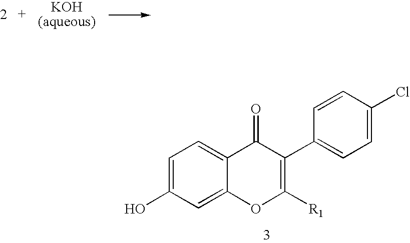

### General Description:

The second part of the second step of Scheme A involves the hydrolysis of an ester compound of formula 2 with aqueous potassium hydroxide to yield a chromen-4-one compound of formula 3.

### B. For Preparing Certain Carbaldehyde Compounds

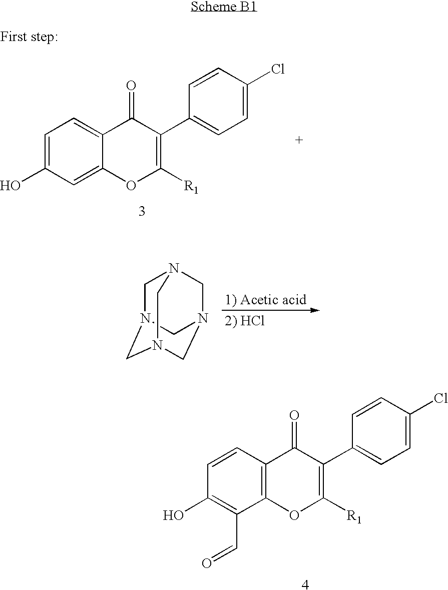

### General Description:

The first step of Scheme B1 involves the reaction of the chromen-4-one compound of formula 3 which was prepared as set forth in Scheme A, with hexamethylenetetramine in the presence of acetic acid to obtain an imine compound which is then reacted with hydrochloric acid to obtain a carbaldehyde compound of formula 4.

### Second Step:

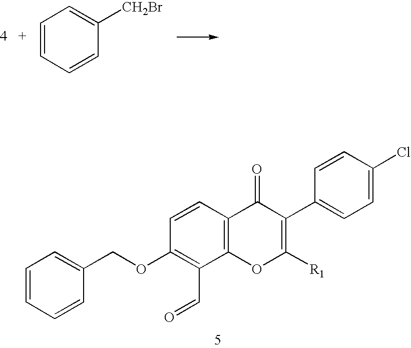

General Description:

The second step of Scheme B1 involves the benzylation of the hydroxy group in a carbaldehyde compound of formula 4 by reacting the latter with benzyl bromide to obtain a benzylated carbaldehyde compound of formula 5.

### C. For Preparing Compounds of Formula (Ia), where R₁ is as Defined Above, R₂ is Chloro, R₃ is Methoxy, R₄ is Hydroxy and m is 1.

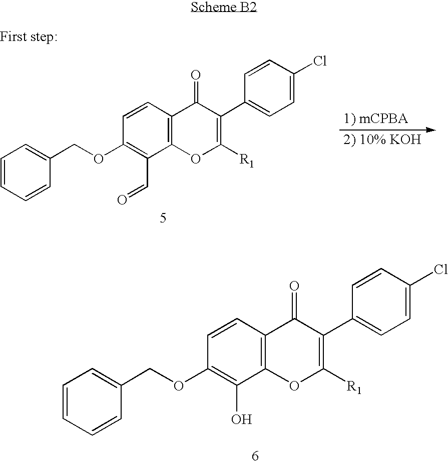

### General Description:

The first step of Scheme B2 involves the oxidation of a benzylated carbaldehyde compound of formula 5 which was prepared as set forth in Scheme B1, with m-chloroperbenzoic acid to obtain an oil which is treated with a 10% KOH solution to yield a chromen-4-one compound of formula 6.

### Second Step:

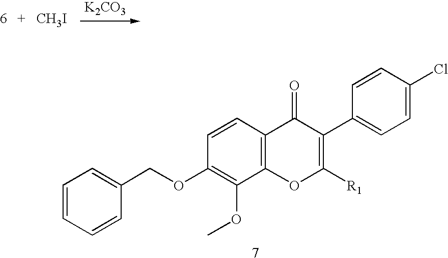

### General Description:

The second step of Scheme B2 involves the alkylation of a chromen-4-one compound of formula 6 with iodomethane in the presence of potassium carbonate to obtain a chromen-4-one compound of formula 7.

### Third Step:

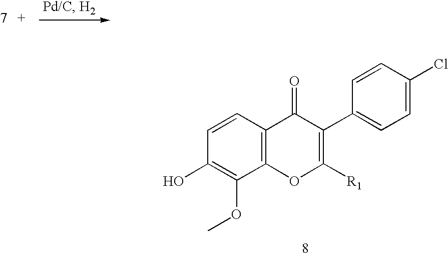

### General Description:

The third step of Scheme B2 involves the debenzylation of a chromen-4-one compound of formula 7 with palladium on carbon in the presence of hydrogen gas to obtain a chromen-4-one compound of formula 8.

### Preparation of Sodium Olate Salt:

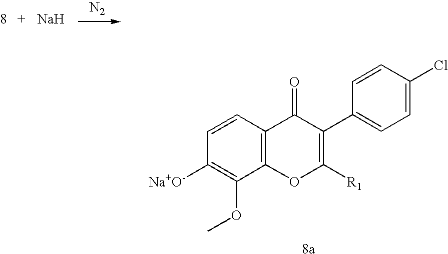

### The sodium olate salt preparation involves the reaction of a chromen-4-one compound of formula 8 with sodium hydride under a nitrogen atmosphere to obtain a corresponding sodium 7-olate compound of formula 8a.

The compounds of formula (Ia), where R₁ is as defined above, R₂ is chloro, R₃ is C₂-C₆alkoxy or (C₃-C₆cycloalkyl)C₁-C₆alkoxy, R₄ is hydroxy and m is 1 can be prepared by utilizing the corresponding ketone compounds which may be prepared by methods disclosed in the literature.

### D. For Preparing Compounds of Formula (Ia), where R₁ is as Defined Above, R₂ is Chloro, R₃ is C₂-C₆alkyl, R₄ is hydroxy and m is 1.

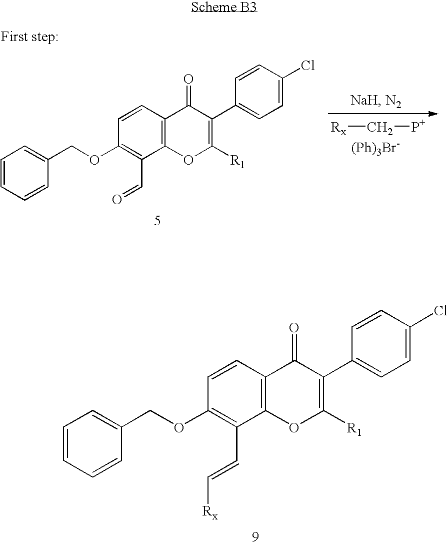

where Rₓ is Hydrogen or C₁-C₄alkyl.

### General Description:

The first step of Scheme B3 involves the Wittig reaction of a carbaldehyde compound of formula 5 which was prepared as set forth in Scheme B1, with a mixture of sodium hydride and an alkyl triphenylphosphonium bromide under a nitrogen atmosphere to obtain an 8-alkenyl substituted chromen-4-one compound of formula 9.

### Second Step:

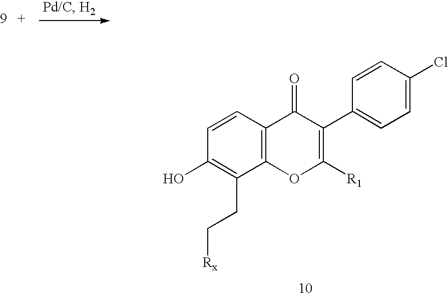

### General Description:

The second step of Scheme B3 involves the debenzylation/hydrogenation of an 8-alkenyl substituted chromen-4-one compound of formula 9 by subjecting it to palladium on carbon in the presence of hydrogen gas to obtain an 8-alkyl substituted chromen-4-one compound of formula 10.

The compounds of formula (Ia), where R₁ is as defined above, R₂ is chloro, R₃ is methyl, R₄ is hydroxy and m is 1 can be prepared by reducing the carbaldehyde compound of formula 5 by methods disclosed in the literature.

### E. For Preparing Compounds of Formula (Ia), where R₁, R₂ and m are as Defined Above, R₃ is Hydrogen and R₄ is Hydroxy.

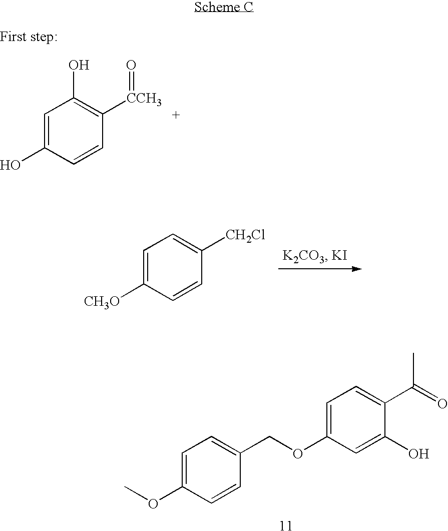

### General Description:

The first step of Scheme C involves the selective alkylation at the 4-position of 2,4-dihydroxyacetophenone with 4-methoxybenzyl chloride in the presence of anhydrous potassium carbonate and potassium iodide to obtain the ethanone compound of formula 11.

### Second Step:

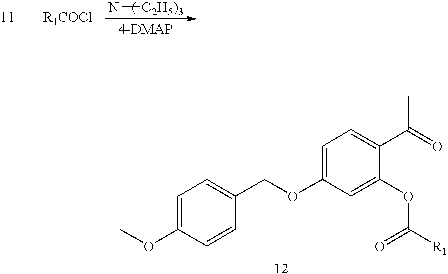

### General Description:

The second step of Scheme C involves the acylation of the ethanone compound of formula 11 with an alkanoyl chloride in the presence of triethylamine and a catalytic amount of 4-dimethylaminopyridine to obtain an ester compound of formula 12.

### Third Step:

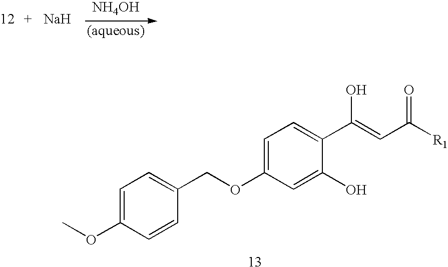

### General Description:

The third step of Scheme C involves the reaction of an ester compound of formula 12 with sodium hydride followed by treatment with aqueous ammonium hydroxide to obtain a compound of formula 13.

### Fourth Step:

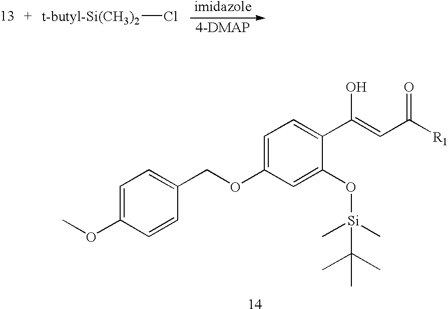

### General Description:

The fourth step of Scheme C involves the selective silylation of the phenolic hydroxy group of a compound of formula 13 by reacting it with t-butyldimethylsilylchloride in the presence of an organic base, e.g., imidazole, and a catalytic amount of 4-dimethylamino pyridine to obtain the corresponding silylised compound of formula 14.

### Fifth Step:

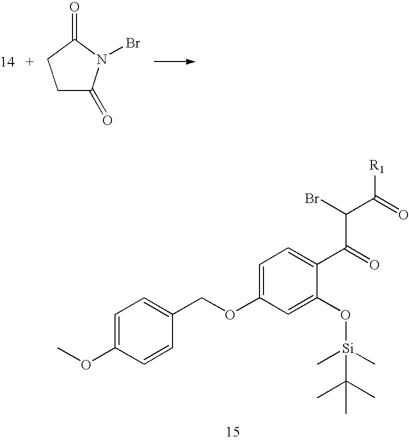

### General Description:

The fifth step of Scheme C involves the reaction of a silylised compound of formula 14 with N-bromosuccinimide to obtain a dione compound of formula 15.

### Sixth Step:

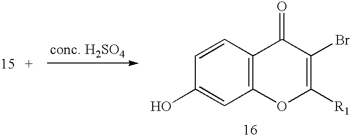

### General Description:

The sixth step of Scheme C involves the desilylisation/cyclisation/debenzylation of a dione compound of formula 15 with concentrated sulfuric acid to obtain a 3-bromo-substituted chromen-4-one compound of formula 16.

### Seventh Step:

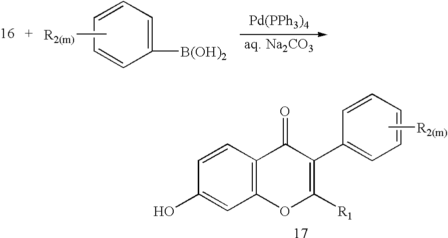

### General Description:

The seventh step of Scheme C involves the Suzuki reaction of a 3-bromo-substituted chromen-4-one compound of formula 16 with a phenyl substituted boronic acid in the presence of a catalytic amount of tetrakis (triphenylphosphine)palladium(0) and aqueous sodium carbonate to obtain a chromen-4-one compound of formula 17.

### F. For preparing compounds of formula (Ia), where R₁ is as defined above, R₂ is chloro, R₃ is hydrogen, R₄ is amino, (C₁-C₆alkyl)amino, a group

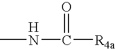

or a group

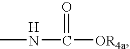

where R₄ₐ is as defined above and m is 1.

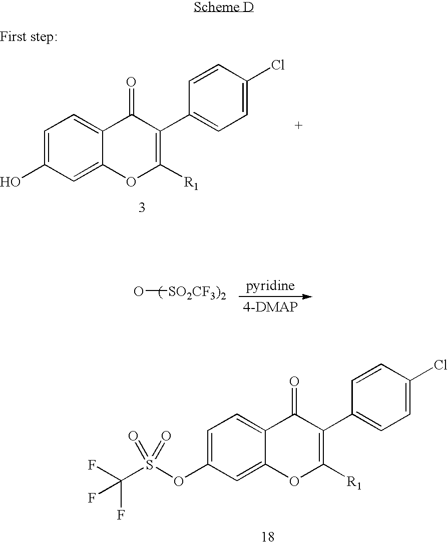

### General Description:

The first step of Scheme D involves the reaction of a chromen-4-one compound of formula 3 which was prepared as set forth in Scheme A, with triflic anhydride in the presence of an organic base, e.g., pyridine, and a catalytic amount of 4-dimethylaminopyridine to obtain a trifluoromethane sulfonic ester compound of formula 18.

### First Part of Second Step:

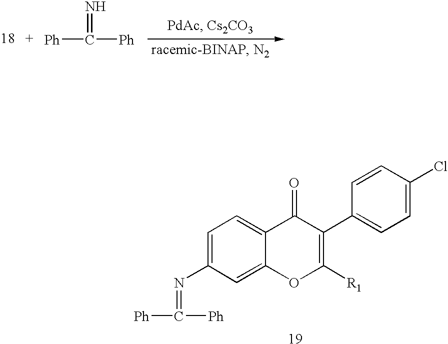

wherein R₁ is as defined above

### General Description:

The first part of the second step of Scheme D involves the reaction of a trifluoromethane sulfonic ester compound of formula 18 with benzophenone imine in the presence of palladium acetate, cesium carbonate and racemic-2,2′-bis(diphenylphosphino)-1,1′-binaphthyl under a nitrogen atmosphere to obtain a 7-benzhydrylidene-substituted chromen-4-one compound of formula 19.

### Second Part of Second Step:

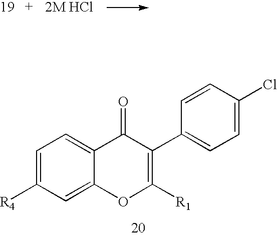

### General Description:

The second part of the second step involves the acid hydrolysis of a 7-benzhydrylidene-substituted chromen-4-one compound of formula 19 with 2M HCl to obtain a chromen-4-one compound of formula 20, where R₄ denotes NH₂ and R₁ is as defined above.

The corresponding alkylamines, amides and carbamates may be prepared by methods described in the literature utilising a compound of formula 20. More particularly, the alkylamines may be prepared by subjecting a compound of formula 20 to reductive alkylation utilising an appropriate aldehyde or ketone. Alternatively, a compound of formula 20 may be reacted with a C₁-C₆alkyl halide. The amides may be prepared by acylating a compound of formula 20 with an appropriate acyl chloride. The carbamates may be prepared by reacting a compound of formula 20 with an appropriate alkylchloroformate.

The starting compounds in Scheme A and Scheme C are known compounds which are commercially available.

Working up the reaction mixtures according to the above processes and purification of the compounds thus obtained may be carried out in accordance with known procedures.

Acid addition salts may be produced from the free bases in known manner, and vice-versa.

Compounds of formulae (I) and (Ia) in optically pure form can be obtained from the corresponding racemates according to well-known procedures, e.g., HPLC with chiral matrix. Alternatively, optically pure starting materials can be used.

Stereoisomeric mixtures, e.g., mixtures of diastereomers, can be separated into their corresponding isomers in a manner known per se by means of suitable separation methods. Diastereomeric mixtures, e.g., may be separated into their individual diastereomers by means of fractionated crystallization, chromatography, solvent distribution and similar procedures. This separation may take place either at the level of a starting compound or in a compound of formula (I) or (Ia) itself. Enantiomers may be separated through the formation of diastereomeric salts, e.g., by salt formation with an enantiomer-pure chiral acid, or by means of chromatography, e.g., by HPLC, using chromatographic substrates with chiral ligands.

In any additional process steps, carried out as desired, functional groups of the starting compounds which should not take part in the reaction may be present in unprotected form or may be protected, e.g., by one or more of the protecting groups mentioned below. The protecting groups are then wholly- or partly-removed according to one of the methods described there.

The protecting groups may already be present in precursors and should protect the functional groups concerned against unwanted secondary reactions. It is a characteristic of protecting groups that they lend themselves readily, i.e., without undesired secondary reactions, to removal, typically by solvolysis, reduction, photolysis or also by enzyme activity, e.g., under conditions analogous to physiological conditions, and that they are not present in the end-products. The skilled artisan knows, or can easily establish, which protecting groups are suitable with the reactions mentioned hereinabove and hereinafter.

The protection of such functional groups by protecting groups, the protecting groups themselves, and their removal reactions are described, e.g., in standard reference works, such as J. F. W. McOmie, Protective Groups in Organic Chemistry, Plenum Press, London and NY (1973); T. W. Greene, Protective Groups in Organic Synthesis, Wiley, NY (1981); The Peptides; Volume 3, E. Gross and J. Meienhofer, Eds., Academic Press, London and NY (1981); Methoden der organischen Chemie (Methods of organic chemistry), Houben Weyl, 4^{th} Edition, Volume 15/1, Georg Thieme Verlag, Stuttgart (1974); H. D. Jakubke and H. Jescheit, Aminosauren, Peptide, Proteine (Amino acids, peptides, proteins), Verlag Chemie, Weinheim, Deerfield Beach, and Basel (1982); and Jochen Lehmann, Chemie der Kohlenhydrate: Monosaccharide und Derivate (Chemistry of carbohydrates: monosaccharides and derivatives), Georg Thieme Verlag., Stuttgart (1974).

All process steps described herein can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralizing agents, e.g., ion exchangers, typically cation exchangers, e.g., in the H⁺ form, depending on the type of reaction and/or reactants at reduced, normal or elevated temperature, e.g., in the range from −100° C. to about 190° C., preferably from about −80° C. to about 150° C., e.g., at −80° C. to 60° C., at room temperature, at −20° C. to 40° C. or at the boiling point of the solvent used, under atmospheric pressure or in a closed vessel, where appropriate under pressure, and/or in an inert atmosphere, e.g., under argon or nitrogen.

Preferred compounds of formula (I) are those wherein

### R₁ is C₁-C₄alkyl, (C₁-C₄alkyl)C₁-C₄alkyl or di-(C₁-C₄alkyl)C₁-C₄alkyl;

### R₂ is chloro, fluoro, trifluoro-substituted C₁-C₄alkyl, C₁-C₄alkylcarbonyl or hydroxy C₁-C₄alkyl;

### R₃ is hydrogen, C₁-C₄alkyl, hydroxy, C₁-C₄alkoxy or (C₃-C₆cycloalkyl)C₁-C₄alkoxy;

### R₄ is hydroxy, amino or (C₁-C₄alkyl)amino;

### R₅ is hydrogen or hydroxy; and

m is 1 or 2.

More preferred compounds of formula (I) are those wherein

### R₁ is C₁-C₄alkyl, (C₁-C₄alkyl)C₁-C₄alkyl or di-(C₁-C₄alkyl)C₁-C₄alkyl;

### R₂ is chloro, fluoro, trifluoromethyl, methylcarbonyl or hydroxymethyl;

### R₃ is hydrogen, C₁-C₄alkyl, hydroxy, C₁-C₄alkoxy or (C₃-C₆cycloalkyl)C₁-C₄alkoxy;

R₄ is hydroxy, amino or (C₁-C₄alkyl)amino;

### R₅ is hydrogen or hydroxy; and

m is 1.

Preferred compounds of formula (Ia) are those wherein

### R₁ is C₁-C₄alkyl, (C₁-C₄alkyl)C₁-C₄alkyl or di-(C₁-C₄alkyl)C₁-C₄alkyl;

### R₂ is chloro, fluoro, trifluoro-substituted C₁-C₄alkyl, C₁-C₄alkylcarbonyl or hydroxy C₁-C₄alkyl;

### R₃ is hydrogen, C₁-C₄alkyl, hydroxy, C₁-C₄alkoxy or (C₃-C₆cycloalkyl)C₁-C₄alkoxy;

### R₄ is hydroxy, amino or (C₁-C₄alkyl)amino; and

m is 1 or 2.

More preferred compounds of formula (Ia) are those wherein

### R₁ is C₁-C₄alkyl, (C₁-C₄alkyl)C₁-C₄alkyl or di-(C₁-C₄alkyl)C₁-C₄alkyl;

### R₂ is chloro, fluoro, trifluoromethyl, methylcarbonyl or hydroxymethyl;

### R₃ is hydrogen, C₁-C₄alkyl, hydroxy, C₁-C₄alkoxy or (C₃-C₆cycloalkyl)C₁-C₄alkoxy;

### R₄ is hydroxy, amino or (C₁-C₄alkyl)amino; and

m is 1.

The even more preferred compounds of the formula I or Ia are the compounds of the Examples, e.g. of the Examples 1 and 3-30.

Another aspect of this invention relates to the fact that the compounds of formulae (I) and (Ia) and their pharmaceutically acceptable salts and, where possible, pharmaceutically acceptable acid addition salts, have beneficial pharmacological activity and, therefore, are useful as pharmaceuticals. In particular, the compounds of formulae (I) and (Ia) exhibit human vanilloid antagonistic activity. More particularly, the compounds of formulae (I) and (Ia) are active at the TRPVI receptor as demonstrated by their ability to inhibit capsaicin and low pH activation of the TRPVI ion channel as follows:

Chinese Hamster Ovary-K1 (CHO-K1) cells, transfected to express either the human, rat or guinea pig TRPV1 receptor, were grown in Minimal Essential Media (MEM) alpha medium without nucleosides supplemented with fetal calf serum (10%), 2 mM L-glutamine, 100 IU/mL penicillin, 100 μg/mL streptomycin and 350-700 μg/mL geneticin. All reagents were supplied by Invitrogen. Cells were grown in T-175 flasks or Costar black, clear-bottomed 96-well view plates and maintained at 37° C. in a 90% humidified incubator with an atmosphere of 5% CO₂ and 95% air. The cells were passaged twice a week at a ratio of 1:10 to 1:20 to maintain steady growth. For experimentation, cells were harvested at approximately 80% confluency and plated onto view plates at 40,000 cells per well in 100 μL media and grown overnight.

### Calcium Mobilisation Assay

On the day of the capsaicin assay, media was aspirated and cells were washed with 100 μL 10 mM N-2-(hydroxyethylpiperazine-N′-[2-ethane-sulfonic acid] (HEPES) buffered Hank's Balanced Salt Solution (HBSS), pH 7.4. Cells were then incubated for 40 minutes with 2.3 μM of the ratiometric calcium binding dye fura-2/AM (from Molecular Probes), made up in HEPES buffered HBSS, containing 0.01% pluronic F-127. For the pH assay, HEPES was omitted and the pH of HBSS adjusted to 7.4. After washing twice with 100 μL assay buffer, cells were incubated for 10 minutes with 100 μL of test compounds (made up in HBSS, pH 7.4), in duplicate, at concentrations between 0.001 and 30 μM. The plate was then placed in a Molecular Devices Flexstation. The TRPV1 receptor was stimulated by application of either capsaicin or low pH. For testing the effect of compounds for possible antagonism, capsaicin was used at the EC₈₀ concentration which was 0.05 μM for the rat TRPV1 receptor, and 0.1 μM for the human and guinea pig. For pH experiments, a low pH buffered solution [60 mM 2-[N-morpholino]ethane sulfonic acid (MES) in HBSS] was added to the assay wells to give a final pH of 5.5.

For determinations of antagonist IC₅₀ values (concentrations of antagonist that inhibit responses to either pH 5.5 or capsacin by 50%), at least 10 antagonist concentrations were measured in duplicate. The response in the presence of the antagonist was calculated as a percentage of the control response to capsaicin or low pH and was plotted against the concentration of antagonist. The IC₅₀ was estimated by non-linear regression analysis to sigmoidal-logistic curves by Activity-Base software (v5.0.10) or Microcal Origin (v7.03). These values were averaged (means and standard error of the mean) for at least three independent experiments.

The compounds of formulae (I) and (Ia), e.g., the compounds of Examples 1 and 3-30, show TRPVI receptor antagonist activity having IC₅₀ values in the range 0.004-30 μM.

In view of the above, the compounds of formulae (I) and (Ia) are useful as vanilloid receptor blockers, e.g., in the treatment of diseases and conditions in which vanilloid receptor activation plays a role or is implicated. Such conditions include, in particular, pain, e.g., bone and joint pain (osteoarthritis), cancer pain, myofascial pain (muscular injury, fibromyalgia) and perioperative pain (general surgery, gynecologic surgery).

The compounds of formulae (I) and (Ia) are particularly useful In the treatment or prevention of chronic pain, especially inflammatory, e.g., chronic inflammatory pain; inflammatory diseases, e.g., inflammatory airways disease, e.g., chronic obstructive pulmonary disease (COPD), or in asthma; cough; urinary incontinence; migraine; visceral disorders, e.g., inflammatory bowel disease; rhinitis; cystitis, e.g. interstitial cystitis; pancreatitis; uveitis; inflammatory skin disorders; and rheumatoid arthritis.

The compounds of formulae (I) and (Ia) are thus useful as vanilloid receptor antagonists, e.g., for the treatment of pain of various genesis or aetiology and as anti-inflammatory and/or anti-edemic agents for the treatment of inflammatory reactions, diseases or conditions, as well as for the treatment of allergic responses. Having regard to their analgesic/anti-inflammatory profile, they are useful for the treatment of inflammatory pain, for the treatment of hyperalgesia and, in particular, for the treatment of severe chronic pain. They are, e.g., useful for the treatment of pain, inflammation and/or oedema consequential to trauma, e.g., associated with burns, sprains, fractures or the like, subsequent to surgical intervention, e.g., as post-operative analgesics, as well as for the treatment of inflammatory pain of diverse genesis, e.g., for the treatment of osteo and rheumatoid arthritis and rheumatic disease, teno-synovitis and gout. They are further suitable as analgesics for the treatment of pain associated with, e.g., angina, menstruation or cancer. As anti-inflammatory/anti-oedema agents, they are further useful, e.g., for the treatment of inflammatory skin disorders, e.g., psoriasis and eczema.

As vanilloid receptor blockers, the compounds of formula (I) and (Ia) are also useful as smooth muscle relaxants, e.g., for the treatment of spasm of the gastrointestinal tract or uterus, e.g., in the therapy of Crohn's disease, ulcerative colitis or pancreatitis.

The compounds of formula (I) and (Ia) are in particular useful as agents for the therapy of airways hyperreactivity and for the treatment of inflammatory events associated with airways disease, in particular, asthma. In addition, the agents of invention may, e.g., be used for the control, restriction or reversal of airways hyperreactivity in asthma.

Inflammatory or obstructive airways diseases to which the present invention is applicable include asthma of whatever type or genesis including both intrinsic and, especially, extrinsic asthma. Thus, the compounds of formula (I) and (Ia) are useful for the treatment of allergic asthma, as well as, e.g., exercise induced asthma, occupational asthma, asthma induced following bacterial infection, other non-allergic asthmas and “wheezy-infant syndrome”.

Efficacy in the treatment of asthma will be evidenced by reduced frequency or severity of symptomatic attack, e.g., of acute asthmatic or bronchoconstrictor attack and by reduced requirement for other, symptomatic therapy, e.g., anti-inflammatory, e.g., corticosteroid; or bronchodilator, e.g., β2 adrenergic, therapy.

Inflammatory or obstructive airways diseases to which the present invention is applicable further include pneumoconiosis (an inflammatory, commonly occupational, disease of the lungs, frequently accompanied by repeated inhalation of dusts) of whatever type or genesis including, e.g., aluminosis, anthracosis, asbestosis, chalicosis, ptilosis, siderosis, silicosis, tabacosis and, in particular, byssinosis.

Further inflammatory or obstructive airways diseases and conditions for which the compounds of formulae (I) and (Ia) may be used include adult respiratory distress syndrome (ARDS), chronic obstructive pulmonary or airways disease (COPD or COAD), and bronchitis. The compounds of formulae (I) and (Ia) may also be used for the treatment of allergic and vasomotor rhinitis.

In addition to the foregoing, the compounds of formulae (I) and (Ia) are also indicated for use in the therapy of septic shock, e.g., as anti-hypovolaemic and/or anti-hypotensive agents; in the treatment of inflammatory bowel disease; cerebral oedema; headache; migraine; inflammatory skin disease, such as eczema and psoriasis; inflammatory disorders of the gut, e.g., irritable bowel syndrome; Crohn's disease; ulcerative colitis; and cystitis, e.g., interstitial cystitis, nephritis and uveitis.

The agents of the invention are useful in the prevention and treatment of diseases and conditions in which human VR1 activation plays a role or is implicated, and therefore susceptible to treatment by the modulation (preferably antagonism) of VR1 receptors. Such conditions include chronic pain with an inflammatory component such as rheumatoid arthritis; bone and joint pain (osteoarthritis); post-surgical pain; musculo-skeletal pain such as fibromyalgia; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, abdominal pain, gynaecological pain, such as dysmenorrhoea, and labour pain; pain associated with the urogenital tract such as cystitis and vulvadynia; inflammatory skin disorders, for example psoriasis and eczema, or itch of non-specific origin; chronic pain associated with nerve injury and/or diseases affecting the nervous system, such as neuropathic pain associated with post-herpetic neuralgia, diabetic neuropathy, chemotherapy-induced neuropathy, amputations (“phantom limb pain”), nerve entrapment and brachial plexus avulsions, low back pain, sciatica and ankylosing spondylitis, reflex sympathetic dystrophy and other chronic nerve injuries; complex regional pain syndromes; central nervous system pain, such as pain due to spinal cord or brain stem damage, or stroke; gout; scar pain; pain associated with carcinoma, often referred to as cancer pain; respiratory diseases including asthma, aluminosis, anthracosis, inflammatory airways disease, e.g. Chronic Obstructive Pulmonary Disease; chronic bronchitis, asbestosis, chalicosis, ptilosis, siderosis, silicosis, tabacosis, byssinosis; rhinitis including allergic rhinitis such as seasonal and perennial rhinitis, and non-allergic rhinitis; cough, either idiopathic or associated with respiratory diseases such as COPD, asthma, cystic fibrosis, cancer, or gastrointestinal disturbances such as gastro-oesophageal reflux; autoimmune diseases; gastrointestinal disorders including but not restricted to irritable bowel syndrome, Crohn's disease, ulcerative colitis, pancreatitis, inflammatory bowel disease. Diseases of the urogenital tract, particularly cystitis; urinary incontinence including bladder detrusor hyper-reflexia and bladder hypersensitivity.

For the above-mentioned indications, the appropriate dosage will of course vary depending upon, e.g., the compound employed, the host, the mode of administration and the nature and severity of the condition being treated. However, in general, satisfactory results in animals are indicated to be obtained at a daily dosage of from about 0.05 to about 150, preferably from about 0.1 mg/kg to about 100 mg/kg animal body weight. In larger mammals, e.g., humans, an indicated daily dosage is in the range from about 0.5 to about 5,000, preferably from about 1 mg to about 500 mg of a compound of formulae (I) and (Ia), conveniently administered, e.g., in divided doses up to four times a day or in sustained-release form.

The compounds of formulae (I) and (Ia) can be administered in vivo either alone or in combination with other pharmaceutical agents effective in the treatment of diseases and conditions in which vanilloid receptor activation plays a role or is implicated including cyclooxygenase-2 (COX-2) inhibitors, such as specific COX-2 inhibitors, e.g., celecoxib and rofecoxib; and non-steroidal anti-inflammatory drugs (NSAIDs), e.g., acetylsalicylic acid and propionic acid derivatives; tricyclic anti-depressants, e.g., Anafranil®, Asendin®, Aventyl®, Elavil®, Endep®, Norfranil®, Norpramin®, Pamelor®, Sinequan®, Surmontil®, Tipramine®, Tofranil®, Vivactil®, Tofranil-PM®; anti-convulsants, e.g., carbamazepine, oxcarbazepine and gabapentin; bradykinin B1 or B2 antagonists; and GABA_{B} agonists, e.g., L-baclofen.

The agents of the invention can be administered in vivo either alone or in combination with other pharmaceutical agents, e.g. agents effective in the treatment of diseases and conditions in which the human VR1 activation plays a role or is implicated, such as cyclooxygenase inhibitors, including specific COX-2 inhibitors (e.g. celecoxib, lumiracoxib, and valdecoxib) or in general nonsteroidal anti-inflammatory drugs (NSAIDs) (e.g. acetylsalicylic acid, propionic acid derivatives), anti-migraine agents such as 5-HTi agonists and CGRP antagonists, tricyclic antidepressants (e.g. clomipramine, amoxapine, nortripyline, amitriptyline, imipramine, desipramine, doxepin, trimipramine, protripyline) selective serotonic reuptake inhibitors (e.g. fluoxetine), selective noradrenaline reuptake inhibitors (e.g. duloxetine), anticonvulsants (e.g. gabapentin, pregabalin, oxcarbazepine, carbamazepine), GABA_{B} agonists (e.g. L-baclofen), opioids (e.g. morphine), CB₁ receptor agonists, bradykinin receptor antagonists, substance P antagonists.

The pharmaceutical compositions for separate administration of the combination partners and for the administration in a fixed combination, i.e., a single galenical composition comprising at least two combination partners, according to the invention can be prepared in a manner known perse and are those suitable for enteral, such as oral or rectal, and parenteral administration to mammals, including man, comprising a therapeutically effective amount of at least one pharmacologically active combination partner alone or in combination with one or more pharmaceutically acceptable carriers, especially suitable for enteral or parenteral application.

Pharmaceutical compositions contain, e.g., from about 0.1% to about 99.9%, preferably from about 20% to about 60%, of the active ingredients. Pharmaceutical preparations for the combination therapy for enteral or parenteral administration are, e.g., those in unit dosage forms, such as tablets including sugar-coated tablets, capsules, suppositories and ampoules. These are prepared in a manner known, per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

A further aspect of the instant invention involves the “novel” compositions comprising a pharmaceutically acceptable carrier or diluent and a therapeutically effective amount of a compound of formula (Ia), in free or salt form and, where possible, in acid addition salt form.

In accordance with the foregoing, the present invention also provides:
(1) A compound of formula (I) or (Ia) in free or salt form and, where possible, in pharmaceutically acceptable acid addition salt form for use as a vanilloid receptor blocker, e.g., for use in any of the particular indications set forth hereinabove;
(2) A compound of formula (I) or (Ia) in free or salt form and, where possible, in pharmaceutically acceptable acid addition salt form for the treatment of a disease or condition in which vanilloid receptor plays a role or is implicated;
(3) A method for the treatment of any of the particular indications set forth hereinabove in a subject in need thereof which comprises administering a therapeutically effective amount of a compound of formula (I) or (Ia) in free or salt form and, where possible, in pharmaceutically acceptable acid addition salt form;
(4) A method for treating or preventing a disease or condition in which vanilloid receptor plays a role or is implicated comprising administering to a mammal in need thereof a therapeutically effective amount of a compound of formula (I) or (Ia) in free or salt form and, where possible, in pharmaceutically acceptable acid addition salt form;
(5) Use of a compound of formula (I) or (Ia) in free or salt form and, where possible, in pharmaceutically acceptable acid addition salt form for the manufacture of a medicament for the treatment or prevention of a disease or condition in which activity of vanilloid receptor plays a role or is implicated;
(6) A method as set forth hereinabove comprising co-administration, e.g., concomitantly or in sequence, of a therapeutically effective amount of a vanilloid receptor antagonist, e.g., a compound of formula (I) or (Ia) in free or salt form and, where possible, in pharmaceutically acceptable acid addition salt form and a second drug substance, said second drug substance being, e.g., for use in any of the particular indications set forth hereinabove; and
(7) A combination comprising a therapeutically effective amount of a compound of formula (I) or (Ia) in free or salt form and, where possible, in pharmaceutically acceptable acid addition salt form and a second drug substance, said second drug substance being, e.g., for use in any of the particular indications set forth hereinabove.

In the Examples which follow, which are not intended to limit, in any way, the scope of the present invention, the following abbreviations are used:
AcOH acetic acid
MeOH methanol
DCM dichloromethane
DMF dimethylformamide
Et₂O diethyl ether
EtOAc ethyl acetate
EtOH ethanol
THF tetrahydrofuran

### EXAMPLE 1

### Preparation of 3-(4-chlorophenyl)-7-hydroxy-2-isopropyl-chromen-4-one (Scheme A)

### a) Preparation of 2-(4-chlorophenyl)-1-(2,4-dihydroxy-phenyl)ethanone

A mixture of resorcinol (100 g, 0.908 mol), 4-chlorophenylacetic acid (170 g, 0.999 mol) and boron trifluoride etherate (587 mL) is stirred mechanically at 85° C. for 1.75 hours. The resultant dark red-brown reaction mixture is allowed to cool to room temperature and then poured slowly into aqueous sodium acetate (1 L, 30% W/v). The resultant suspension is stirred overnight at room temperature. The resultant orange brown precipitate is removed by filtration, dried in vacuo and then triturated with isopropyl ether/hexane (1:9 ratio) to give a yellow solid. The yellow solid is washed with hexane and dried in vacuo to give the desired compound. A further three crops of material are obtained from the sodium acetate work-up mixture.

### b) Preparation of isobutyric acid 3-(4-chlorophenyl)-2-isopropyl-4-oxo-4H-chromen-7-yl ester

A mixture of the compound prepared in Example 1a above (100 g, 0.382 mol) iso-butyric anhydride (380 mL, 2.29 mol) and dry pyridine (380 mL, 4.69 mol) is stirred at 140° C. for 12 hours and then allowed to cool to room temperature. The volatile components are removed in vacuo and the resulting dark brown oil is dried under high vacuum to give the crude compound.

### c) Preparation of the Title Compound

To a mixture of the compound prepared in Example 1b above and MeOH (400 mL) is added aqueous KOH (250 mL, 5M) which resulted in a rather high exotherm. The resultant dark solution is stirred for 1.5 hours and the MeOH is then evaporated in vacuo. The resulting solution is acidified with 2M HCl to pH 3 to give a brown precipitate, which is removed by filtration. The resultant brown solid is washed with water (3×), isopropyl ether and then air-dried. The remaining aqueous solution is extracted with EtOAc (4×) and the combined organic phases are washed with water (3×), dried (Na₂SO₄) and evaporated to give a red oil, which solidifies to give a brown solid. The brown solid is washed with isopropyl ether and air-dried. The combined aqueous phases are extracted again (EtOAc) to provide a third crop of product.

¹H NMR (400 MHz, DMSO-d₆): δ 7.86 (1H, d, J=8.7 Hz), 7.50 (2H, d, J=9.0 Hz), 7.27 (2H, d, J=9.0 Hz), 6.90 (1H, dd, J=2.2, 8.6 Hz), 6.86 (1H, d, J=2.2 Hz), 2.77 (1H, quint, J=6.9 Hz), 1.19 (6H, d, J=6.9 Hz); (M+H)⁺=316.0; HPLC retention time=5.1 minutes.

### EXAMPLE 2

### Preparation of 7-benzyloxy-3-(4-chlorophenyl)-2-isopropyl-4-oxo-4H-chromen-8-carbaldehyde (Scheme B1)

### a) Preparation of 3-(4-chlorophenyl)-7-hydroxy-2-isopropyl-4-oxo-4H-chromen-8-carbaldehyde

A mixture of the compound of Example 1 (12.48 g, 39.6 mmol) and hexamethylenetetramine (39.46 g, 0.28 mol) in AcOH (250 mL) is stirred at 100° C. for 20 hours. After the mixture cools to room temperature, the solvent is removed in vacuo to afford a black oily residue. 5M HCl solution (150 mL) is added and the resultant mixture is heated under reflux for 30 minutes. The reaction mixture is then poured onto ice/water and the resulting brown solid is isolated by filtration. The solid is then taken up in CH₂Cl₂, passed through a bed of Celite, and the solvent is evaporated in vacuo. The resultant solid residue is stirred at room temperature with EtOAc, filtered and washed with hexane to afford the desired product as a pale brown solid (7.06 g, 52%).

¹H NMR (400 MHz, DMSO-d₆): δ 10.6 (1H, s), 8.2 (1H, dd, J=2.8, 8.96 Hz), 7.56 (2H, dd, J=2.7, 8.4 Hz), 7.34 (2H, dd, J=2.9, 8.5 Hz), 7.14 (1H, dd, J=2.7, 8.96 Hz), 2.84 (1H, quint, J=6.8 Hz), 1.32-1.29 (6H, d, J=6.8 Hz).

### b) Preparation of the Title Compound

To a solution of the compound prepared in Example 2a above (7.95 g, 23.2 mmol) and benzyl bromide (7.93 g, 46.4 mmol) in DMF (200 mL) is added K₂CO₃ (9.61 g, 69.5 mmol), and the reaction mixture is stirred at room temperature for 96 hours. The mixture is then poured into ice/water, extracted with CH₂Cl₂, dried (MgSO₄) and concentrated in vacuo. The resulting solid residue is stirred with hexane/EtOAc for 1 hour, the solvent is decanted and the solid is stirred with hexane/Et₂O for 16 hours. The title compound is collected by filtration and washed with hexane to afford a pale brown solid.

¹H NMR (400 MHz, DMSO-d₆): δ 10.6 (1H, s), 8.26 (1H, d, J=9 Hz), 7.58-7.35 (8H, m), 7.29 (2H, d, J=8.4 Hz), 5.47 (2H, s), 2.78 (1H, quint, J=6.8 Hz), 1.26-1.24 (6H, d, J=6.8 Hz); (M+H)⁺=433.3; HPLC retention time=7.1 minutes.

### EXAMPLE 3

### Preparation of 3-(4-chlorophenyl)-7-hydroxy-2-isopropyl-8-methoxy-chromen-4-one (Scheme B2)

### a) Preparation of 7-benzyloxy-3-(4-chlorophenyl)-8-hydroxy-2-isopropyl-chromen-4-one

To a solution of the compound of Example 2 (8.03 g, 18.6 mmol) in CH₂Cl₂ (200 mL) is added mCPBA (9.24 g, 53.5 mmol). The reaction mixture is stirred at 50° C. for 4 hours and washed with a saturated NaHCO₃ solution. The solution is dried (MgSO₄) and concentrated in vacuo to give a yellow oil.

To a solution of the oil in MeOH (350 mL) is added a 10% KOH solution (35 mL) and the mixture is stirred at room temperature overnight. The solvent is concentrated to a volume of 50 mL, ice/water is added and the solution is acidified with concentrated HCl. A white solid is isolated by filtration, washed with water and taken up into CH₂Cl₂. The CH₂Cl₂ solution is dried (MgSO₄) and the solvent is removed in vacuo to afford a dark brown solid. The solid is stirred in hot hexane/EtOAc, and filtered to afford the desired compound as a white solid.

¹H NMR (400 MHz, DMSO-d₆): δ 9.57 (1H, s, exchanges with D₂O), 7.52-7.21 (11H, m), 5.34 (2H, s), 2.78 (1H, quint, J=6.9 Hz), 1.25-1.23 (6H, d, J=6.8 Hz).

### b) Preparation of 7-benzyloxy-3-(4-chlorophenyl)-2-isopropyl-8-methoxy-chromen-4-one

To a solution of the compound prepared in Example 3a above (3.01 g, 7.15 mmol) and iodomethane (1.17 g, 8.22 mmol) in DMF (60 mL) is added K₂CO₃ (1.98 g, 14.3 mmol), and the reaction mixture is stirred at room temperature for 72 hours. The mixture is diluted with EtOAc and water, and the organic phase is washed with sodium thiosulfate solution, brine, dried (MgSO₄) and concentrated in vacuo. The resulting off-white solid residue is triturated with EtOAc to afford the desired compound as a white solid.

¹H NMR (400 MHz, DMSO-d₆): δ 7.73 (1H, d, J=8.98 Hz), 7.5 (4H, d, J=8.3 Hz), 7.43 (2H, t, J=7.7 Hz), 7.36 (2H, t, J=7.2 Hz), 7.29 (2H, d, J=8.8 Hz), 5.34 (2H, s), 3.93 (3H, s), 2.81 (1H, quint, J=6.8 Hz), 1.25-1.23 (6H, d, J=6.8 Hz).

### c) Preparation of the Title Compound

A suspension of the compound prepared in Example 3b above (2.68 g, 6.16 mmol) and 20% Pd/carbon (268 mg) in THF (30 mL), absolute EtOH (30 mL) and 5M HCl solution (15 mL) is stirred under a balloon of H₂ at room temperature for 3 hours. The reaction mixture is filtered through a pad of Celite filter aid, which is itself washed with THF. The solvent is removed under reduced pressure to afford the desired compound.

¹H NMR (400 MHz, DMSO-d₆): δ10.6 (1H, br, s, exchanges with D₂O), 7.67 (1H, d, J=8.8 Hz), 7.55 (2H, d, J=8.3 Hz), 7.33 (2H, d, J=8.3 Hz), 7.05 (1H, d, J=8.8 Hz), 3.96 (3H, s), 2.86 (1H, quint, J=6.8 Hz), 1.3-1.28 (6H, d, J=6.8 Hz); (M+H)⁺=345.2; HPLC retention time=5.1 minutes.

### d) Preparation of the Sodium Olate Salt of the Title Compound

A solution of the compound prepared in Example 3c above (46.6 mg, 0.135 mmol) in dry THF (1 mL) is treated with sodium hydride (7.57 mg, 0.189 mmol, 60% dispersion in mineral oil). The mixture is stirred under N₂ at room temperature for 30 minutes and the solvent is then removed under reduced pressure. The residue is re-suspended in CHCl₃ and the solvent is removed in vacuo. This procedure is repeated twice more to afford the desired compound.

¹H NMR (400 MHz, DMSO-d₆): δ 7.53 (2H, d, J=8.35 Hz), 7.41 (1H, d, J=8.96 Hz), 7.31 (2H, d, J=8.4 Hz), 6.54 (1H, d, J=8.9 Hz), 3.84 (3H, s), 2.82 (1H, quint, J=6.8 Hz), 1.29-1.27 (6H, d, J=6.8 Hz); (M+H)⁺=345.0; HPLC retention time=5.1 minutes.

### EXAMPLE 4

### Preparation of 3-(4-chlorophenyl)-7-hydroxy-2-isopropyl-8-propyl-chromen-4-one (Scheme B3)

### a) Preparation of 7-benzyloxy-3-(4-chlorophenyl)-2-isopropyl-8-propenyl-chromen-4-one

To a mixture of sodium hydride (149 mg, 3.74 mmol, 60% dispersion in mineral oil) in dry THF (30 mL) under N₂ is added portionwise over 10 minutes, ethyltriphenylphosphonium bromide (1.39 g, 3.74 mmol). The resultant mixture is stirred at room temperature for 30 minutes, becoming a pale yellow solution. To this solution is slowly added a solution of the compound of Example 2 (900 mg, 2.08 mmol) in dry THF (8 mL), and the resultant solution is stirred at room temperature for 5 hours. The solution is then diluted with water, extracted twice into CH₂Cl₂ and dried over anhydrous MgSO₄. Removal of solvent under reduced pressure afforded a yellow oil which is purified by flash chromatography over silica gel (10% EtOAc/hexane) to afford the desired compound as a 1:1 mixture of cis and trans isomers (white foam).

¹H NMR (400 MHz, DMSO-d₆): δ 8.03 (1H, d, J=8.9 Hz), 7.93 (1H, d, J=8.9 Hz), 7.6-7.3 (20H, m), 6.76 (2H, d, J=2.4 Hz), 6.4 (1H, dd, J=1.6, 11.2 Hz), 6.12 (1H, dd, J=6.8, 11.2 Hz), 5.42 (2H, s), 5.37 (2H, s), 2.87 (2H, quint, J=6.8 Hz), 2.02 (3H, dd, J=2.3, 4.6 Hz), 1.61 (3H, dd, J=1.7, 6.8 Hz), 1.3-1.28 (6H, d, J=6.8 Hz), 1.24-1.23 (6H, d, J=6.8 Hz).

### b) Preparation of the Title Compound

A suspension of the compound prepared in Example 4a above (78.3 mg, 1.76 mmol) and 20% Pd/carbon (157 mg) in THF (6 mL), absolute EtOH (6 mL) and 5M HCl solution (3 mL) is stirred under a balloon of H₂ at room temperature for 5 hours. The reaction mixture is filtered through a pad of Celite filter aid, which is itself washed with EtOH and EtOAc. The solvent is removed under reduced pressure and the title compound is precipitated as a cream-coloured solid by dissolution of the residue in EtOAc and addition of hexane.

¹H NMR (400 MHz, DMSO-d₆): δ10.61 (1H, s, exchanges with D₂O), 7.8 (1H, d, J=8.7 Hz), 7.55 (2H, d, J=8.4 Hz), 7.34 (2H, d, J=8.3 Hz), 7.03 (1H, d, J=8.7 Hz), 2.9-2.8 (3H, m), 1.75-1.6 (2H, m), 1.3-1.28 (6H, d, J=6.8 Hz), 1.03 (3H, t, J=7.4 Hz); (M+H)⁺=357.0; HPLC retention time=6.5 minutes.

### EXAMPLE 5

### Preparation of 3-(4-fluorophenyl)-7-hydroxy-2-isopropyl-chromen-4-one (Scheme C)

### a) Preparation of 1-[2-hydroxy-4-(4-methoxy-benzyloxy)-phenyl]-ethanone

A mixture of 2′,4′-dihydroxyacetophenone (11.71 g, 0.077 mol), 4-methoxybenzyl chloride (10.44 mL, 0.077 mol), anhydrous potassium carbonate (11.75 g, 0.085 mol) and potassium iodide (12.78 g, 0.077 mol) are heated together in refluxing dry acetone (80 mL) for 4 hours. The mixture is then cool to room temperature, poured into water (250 mL) and extracted with EtOAc (3×100 mL). The EtOAc extracts are combined, washed with saturated brine (100 mL), dried (MgSO₄), filtered and concentrated until crystallization commences. After standing at 4° C. for 16 hours, the crystals are recovered by filtration, washed with cold EtOAc and then with n-hexane and dried to yield the desired compound.

### b) Preparation of isobutyric acid 2-acetyl-5-(4-methoxy-benzyloxy)-phenyl ester

The compound prepared in Example 5a above (9.11 g, 0.034 mol) is dissolved in dry DCM (120 mL) under an atmosphere of dry argon. Triethylamine (5.14 mL, 0.037 mol) and 4-dimethylaminopyridine (0.204 g, 1.67 mmol) are added and the resultant mixture is cooled to 0° C. using an ice-water bath. Isobutyryl chloride (3.89 mL, 0.037 mol) is then added dropwise and the mixture is stirred while warming to room temperature. The mixture is then poured into water (100 mL) and the DCM layer is separated, washed with saturated brine (100 mL), dried (MgSO₄), treated with activated charcoal (300 mg), filtered and evaporated to yield the desired compound as a pale pink solid.

### c) Preparation of 1-hydroxy-1-[2-hydroxy-4-(4-methoxy-benzyloxy)-phenyl]-4-methyl-pent-1-en-3-one (and keto tautomer)

To a solution of the compound prepared in Example 5b above (11.45 g, 0.033 mol) in dry THF (160 mL) is added, portionwise over a period of ˜15 minutes at room temperature, sodium hydride (60% dispersion on mineral oil, 4.68 g, 0.117 mol). The reaction mixture is stirred at room temperature for 2 hours during which there was a slight exotherm and the mixture reached a temperature of ˜40° C. Aqueous 5% ammonium hydroxide (100 mL) is then carefully added to quench the reaction and then the mixture is poured into water (200 mL) and extracted with EtOAc (3×75 mL). The EtOAc extracts are combined, washed with saturated brine (100 mL), dried (Na₂SO₄), filtered and concentrated under reduced pressure until crystallization commences. After standing at 4° C. for 16 hours, the crystals are recovered by filtration, washed with n-hexane and dried to yield the desired compound.

### d) Preparation of 1-[2-tert-butyl-dimethyl-silanyloxy)-4-(4-methoxy-benzyloxy)-phenyl]-1-hydroxy-4-methyl-pent-1-en-3-one (and keto tautomer)

The compound prepared in Example 5c above (4.75 g, 13.9 mmol), t-butyldimethylsilylchloride (2.3 g, 15.3 mmol), imidazole (1.04 g, 15.3 mmol) and 4-dimethylaminopyridine (0.17 g, 1.4 mmol) are mixed together in dry DMF (100 mL) at room temperature under argon for 60 hours. The resultant mixture is poured into water (300 mL) and extracted with diethyl ether (3×100 mL). The ether extracts are combined, washed with saturated brine (100 mL), dried (MgSO₄), filtered and evaporated to give a cream coloured solid. This solid is then re-crystallized from hot n-hexane to yield the desired compound as a colourless crystalline solid. If desired, additional product could be obtained by chromatography (silica gel) of the residues from the mother liquor using cyclohexane and cyclohexane/EtOAc (4:1) as eluant.

¹H NMR (400 MHz, CDCl₃): δ 7.74 (1H, d, J=8.8 Hz), 7.34 (2H, d, J=8.7 Hz), 6.92 (2H, d, J=8.7 Hz), 6.66 (1H, dd, J=2.4, 8.8 Hz), 6.41 (1H, d, J=2.4 Hz), 6.34 (1H, s), 5.00 (2H, s), 3.82 (3H, s), 2.53 (1H, m), 1.18 (6H, d, J=6.9 Hz), 0.98 (9H, s), 0.21 (6H, s).

### e) Preparation of 2-bromo-1-[2-(tert-butyl-dimethyl-silanyloxy)-4-(4-methoxy-benzyloxy)-phenyl]-4-methyl-pentane-1,3-dione

The compound prepared in Example 5d above (5.81 g, 12.72 mmol) is dissolved in dry DCM (100 mL) at room temperature and N-bromosuccinimide (2.38 g, 13.36 mmol) is added portionwise. The reaction mixture is stirred at room temperature for 30 minutes, poured into water (200 mL) and extracted with DCM (3×75 mL). The DCM extracts are combined, washed with saturated brine (100 mL), dried (MgSO₄), filtered and evaporated to yield the desired compound as a pale yellow solid.

### f) Preparation of 3-bromo-7-hydroxy-2-isopropyl-chromen-4-one

The compound prepared in Example 5e above (6.77 g, 12.65 mmol) is dissolved in absolute EtOH (350 mL) at 50° C. and concentrated sulfuric acid (16 mL) is added dropwise. The resultant mixture is stirred at 50° C. for 16 hours, after which an additional 0.5 mL concentrated sulfuric acid is added and stirring is continued for a further 4 hours at 50° C. The reaction mixture is cooled to room temperature and most of the EtOH is removed under reduced pressure. Water (400 mL) is added to the residue and the colourless solid formed is recovered by filtration and dried in a desiccator. Since the product is not pure enough for subsequent use, it is partitioned between water and EtOAc and extracted with EtOAc (3×100 mL). The EtOAc extracts are combined, washed with saturated brine (100 mL), dried (MgSO₄), treated with activated charcoal (300 mg), filtered and concentrated until crystallization commences. After standing at 4° C. for 16 hours, the crystals are recovered by filtration, washed with n-hexane and dried to yield the desired compound.

¹H NMR (400 MHz, DMSO): δ 10.89 (0.8H, br, s, partially exchanged), 7.89 (1H, d, J=8.8 Hz), 6.94 (1H, dd, J=2.2, 8.8 Hz), 6.87 (1H, d, J=2.2 Hz), 3.50 (1H, m), 1.28 (6H, d, J=6.9 Hz).

### g) Preparation of the Title Compound

The compound prepared in Example 5f above (105 mg, 0.371 mmol), 4-fluorobenzene-boronic acid (83 mg, 0.593 mmol) and tetrakis (triphenylphosphine) palladium(0) (22 mg, 0.019 mmol) are dissolved in EtOH (4.5 mL) in a 5 mL Personal Chemistry microwave tube. Aqueous sodium carbonate solution (2M, 0.5 mL) is added and the tube is sealed. The mixture is heated at 130° C. for 20 minutes in a Personal Chemistry Emrys Optimiser microwave instrument. After cooling to room temperature, the mixture is partitioned between EtOAc and water and extracted with EtOAc (3×20 mL). The EtOAc extracts are combined, washed with saturated brine (50 mL), dried (MgSO₄), treated with activated charcoal (100 mg), filtered and evaporated under reduced pressure to yield the title compound as a pale yellow solid.

¹H NMR (400 MHz, DMSO): δ 7.86 (1H, d, J=8.7 Hz), 7.28 (4H, m), 6.90 (1H, dd, J=2.2, 8.7 Hz), 6.86 (1H, d, J=2.2 Hz), 2.77 (1H, m), 1.19 (6H, d, J=6.8 Hz); (M+H)⁺=299.2; HPLC retention time=4.6 minutes.

### EXAMPLE 6

### Preparation of 7-amino-3-(4-chlorophenyl)-2-isopropyl-chromen-4-one (Scheme D)

### a) Preparation of trifluoromethanesulfonic acid 3-(4-chlorophenyl)-2-isopropyl-4-oxo-4H-chromen-7-yl ester

A mixture of the compound of Example 1 (5.11 g, 16.2 mmol), DMAP (0.198 g, 1.62 mmol) and pyridine (5.5 g, 70 mmol) in anhydrous CH₂Cl₂ (170 mL) is cooled in an ice bath. A solution of triflic anhydride (9.0 g, 32 mmol) in anhydrous CH₂Cl₂ (10 mL) is added, dropwise, to the reaction mixture which is allowed to warm to room temperature over 3 hours. 1M HCl solution (150 mL) is added, the resultant mixture is stirred for 10 minutes and the two phases are separated. The aqueous phase is washed with CH₂Cl₂ (3×). The organic phases are combined, dried (MgSO₄) and the solvent is removed under reduced pressure. The resulting red oil is dried in vacuo to afford the desired compound as a pink foam.

¹H NMR (400 MHz, DMSO-d₆): δ 8.21 (1H, d, J=8.8 Hz), 8.09 (1H, d, J=2.4 Hz), 7.62 (1H, dd, J=2.4, 8.8 Hz), 7.53 (2H, d, J=8.5 Hz), 7.31 (2H, d, J=8.5 Hz), 2.82 (1H, quint, J=6.9 Hz), 1.25-1.23 (6H, d, J=6.8 Hz).

### b) Preparation of 7-(benzhydrylideneamino)-3-(4-chlorophenyl)-2-isopropyl-chromen-4-one

A mixture of the compound prepared in Example 6a above (6.96 g, 15.6 mmol), palladium acetate (0.35 g, 1.56 mmol), cesium carbonate (12.7 g, 38.9 mmol) and racemic-BINAP (0.97 g, 1.56 mmol) in anhydrous THF (230 mL) under an atmosphere of nitrogen is treated with benzophenone imine (3.66 g, 20.2 mmol) and allowed to stir 80° C. for 22 hours. After allowing the resultant mixture to stir at room temperature for an additional 24 hours, it is diluted with water (300 mL) and extracted with EtOAc (3×300 mL). The organic extracts are combined, washed with brine, dried (MgSO₄), filtered, concentrated in vacuo and purified by flash chromatography over silica gel (10% EtOAc/cyclohexane) to afford the desired compound as a dark yellow solid.

### c) Preparation of the Title Compound

A solution of the compound prepared in Example 6b above (5.72 g, 12 mmol) in THF (150 mL) is treated with 2M HCl solution (150 mL) and allowed to stir at room temperature for 1 hour. The solution is basified with 17% ammonia solution (150 mL) and extracted with EtOAc (3×200 mL). The organic extracts are combined, dried (MgSO₄), filtered and concentrated to afford a yellow suspension. The suspension is triturated with hexanes to afford the title compound as a pale yellow solid which is isolated by filtration and dried in vacuo overnight.

¹H NMR (400 MHz, DMSO-d₆): δ 7.67 (1H, d, J=8.7 Hz), 7.47 (2H, d, J=8.4 Hz), 7.25 (2H, d, J=8.4 Hz), 6.66 (1H, dd, J=2.0, 8.7 Hz), 6.52 (1H, d, J=2.0 Hz), 6.25 (2H, s, exchanges with D₂O), 2.72 (1H, quint, J=6.8 Hz), 1.19-1.17 (6H, d, J=6.8 Hz); (M+H)⁺=314.2, HPLC retention time=5.0 minutes.

### EXAMPLES 7 TO 30

The compounds of Examples 7 to 30 can be prepared in a manner analogous to that described in the previous Examples.

| | | | HPLC | |
|---|---|---|---|---|
| | | | Retention | |
| | | | Time | Method of |
| Example | Structure | (M + H)⁺ | (minutes) | Preparation |
| 7 | 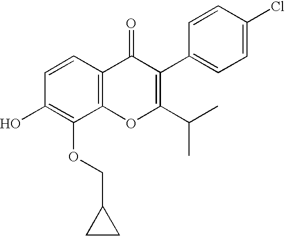 | 385.2 | 5.9 | A + B1 + B2 |
| 8 | 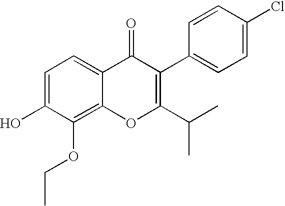 | 359.0 | 5.6 | A + B1 + B2 |
| 9 | 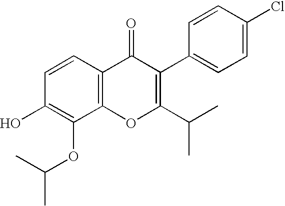 | 373.3 | 5.9 | A + B1 + B2 |
| 10 | 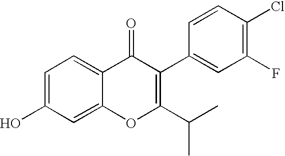 | 333.2 | 5.2 | C |
| 11 | 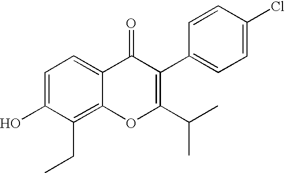 | 343.0 | 6.1 | A + B1 + B3 |
| 12 | 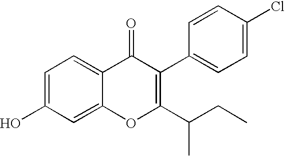 | 329.9 | 5.5 | A |
| 13 | 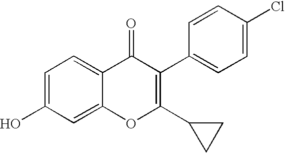 | 313.2 | 4.8 | A |
| 14 | 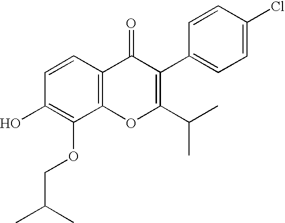 | 387.2 | 6.6 | A + B1 + B2 |
| 15 | 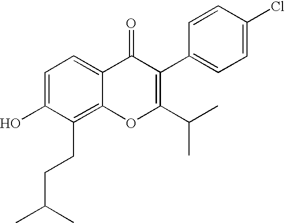 | 385.0 | 7.3 | A + B1 + B3 |
| 16 | 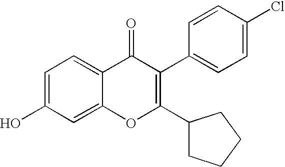 | 341.2 | 5.7 | A |
| 17 | 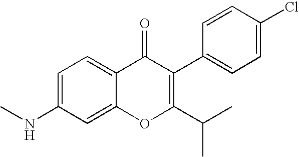 | 328.2 | 5.7 | A + D |
| 18 | 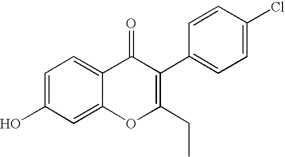 | 301.2 | 4.7 | A |
| 19 | 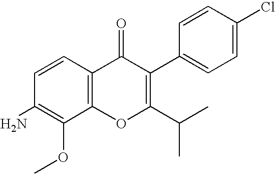 | 344.0 | 5.2 | A + B1 + B2 + D |
| 20 | 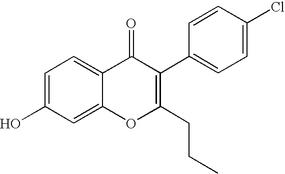 | 315.2 | 5.2 | A |
| 21 | 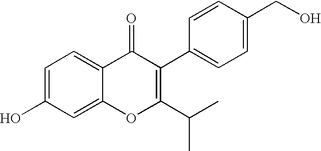 | 311.3 | 3.3 | C |
| 22 | 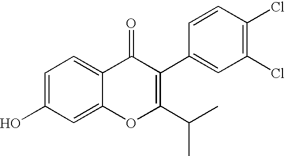 | 350.0 | 5.6 | C |
| 23 | 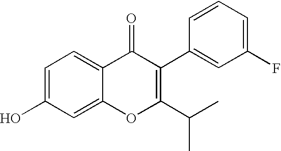 | 299.2 | 4.6 | C |
| 24 | 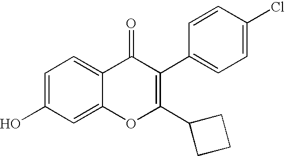 | 327.2 | 5.4 | A |
| 25 | 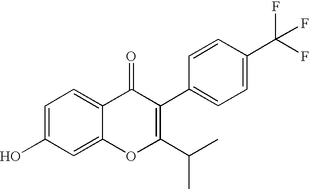 | 349.2 | 5.4 | C |
| 26 | 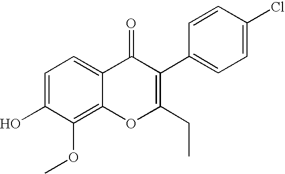 | 331.1 | 4.7 | A + B1 + B2 |
| 27 | 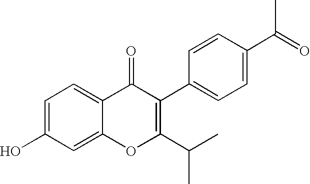 | 323.2 | 4.1 | C |
| 28 | 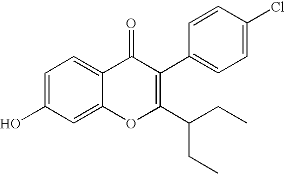 | 343.2 | 5.9 | A |
| 29 | 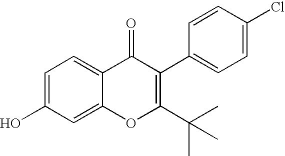 | 329.2 | 5.3 | A |
| 30 | 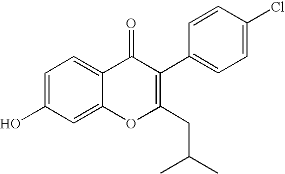 | 329.2 | 5.5 | A |

### EXAMPLE 31

The compounds 31.1 to 31.79 can be prepared in a manner analogous to that described in the previous Examples.

| | | HPLC | |
|---|---|---|---|
| | | Retention | |
| | | Time | |
| No. | Structure | (in minutes) | (M + H)⁺ |
| 31.1 | 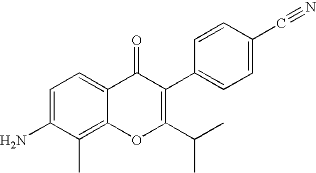 | | 431.3 |
| 31.2 | 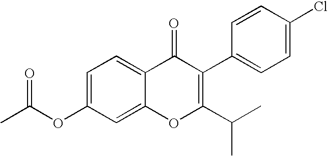 | 6.1 | 357.0 |
| 31.3 | 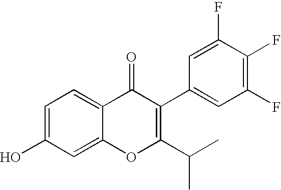 | 5.1 | 335.0 |
| 31.4 | 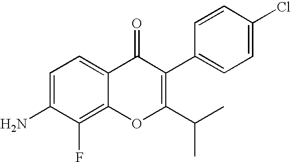 | 5.6 | 332.8 |
| 31.5 | 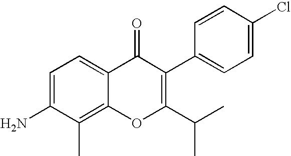 | | 440.7 |
| 31.6 | 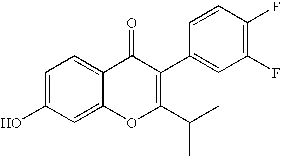 | 4.8 | 317.0 |
| 31.7 | 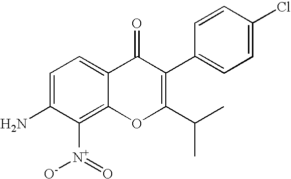 | | 359.8 |
| 31.8 | 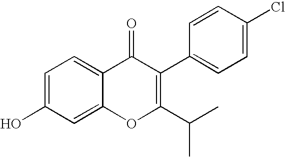 | 5.1 | 315.0 |
| 31.9 | 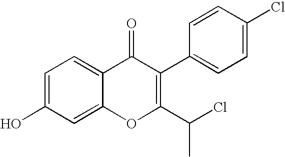 | 5.1 | 337.8 |
| 31.10 | 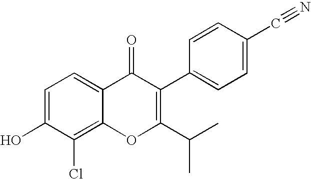 | | 340.8 |
| 31.11 | 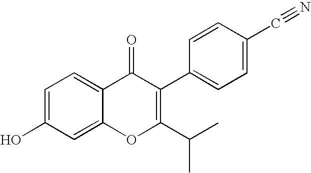 | | 306.3 |
| 31.12 | 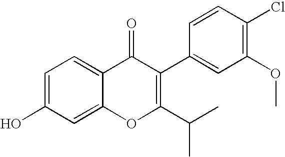 | 5.0 | 345.0 |
| 31.13 | 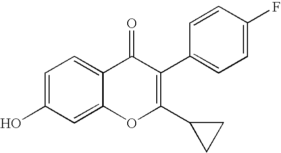 | 4.3 | 297.8 |
| 31.14 | 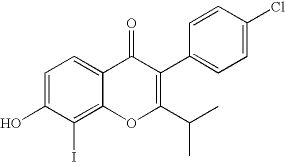 | | 441.7 |
| 31.15 | 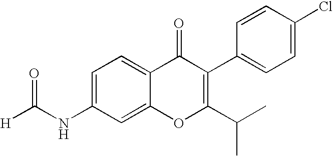 | | 342.2 |
| 31.16 | 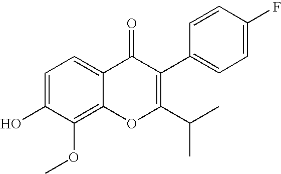 | 4.6 | 329.2 |
| 31.17 | 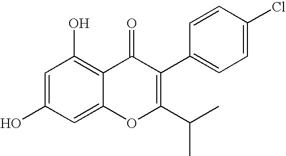 | 5.9 | 330.9 |
| 31.18 | 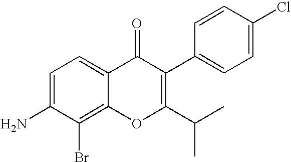 | 6.1 | 393.4 |
| 31.19 | 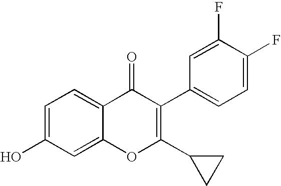 | 4.5 | 314.9 |
| 31.20 | 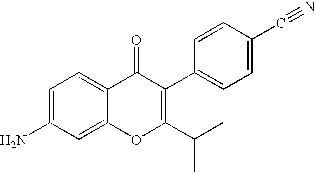 | | 305.4 |
| 31.21 | 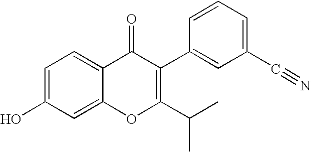 | 4.2 | 306.0 |
| 31.22 | 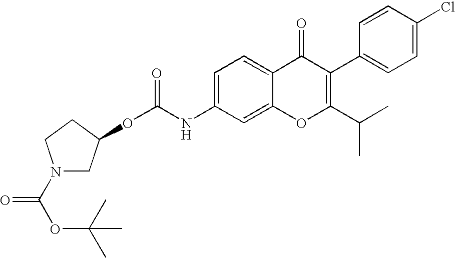 | 6.9 | 527.4 |
| 31.23 | 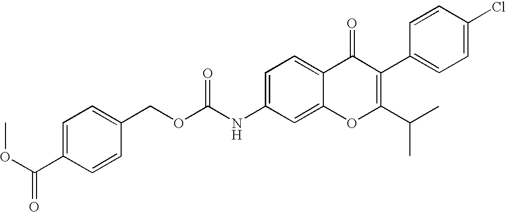 | 7.0 | 506.0 |
| 31.24 | 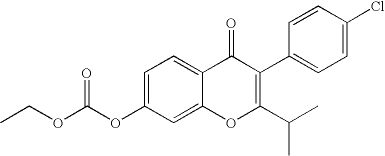 | 6.7 | 387.0 |
| 31.25 | 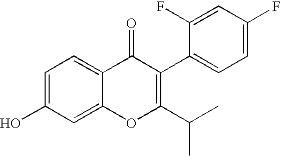 | | 317.3 |
| 31.26 | 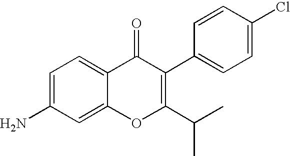 | 5.1 | 314.0 |
| 31.27 | 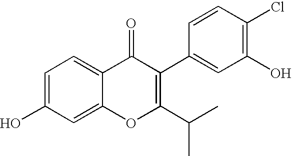 | 4.2 | 331.0 |
| 31.28 | 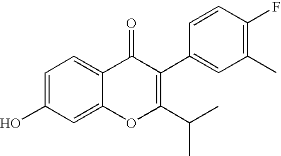 | | 313.3 |
| 31.29 | 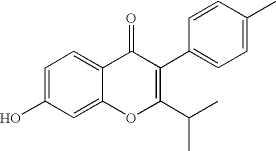 | 5.0 | 295.0 |
| 31.30 | 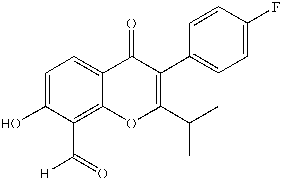 | 5.5 | 327.3 |
| 31.31 | 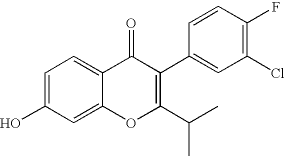 | | 333.8 |
| 31.32 | 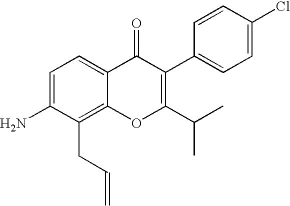 | 8.4 | 354.2 |
| 31.33 | 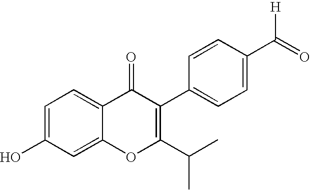 | | 310.0 |
| 31.34 | 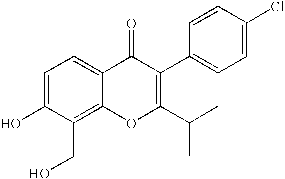 | 7.4 | 345.2 |
| 31.35 | 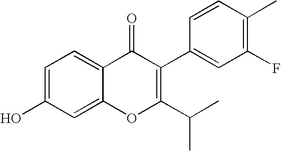 | | 313.3 |
| 31.36 | 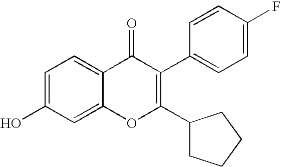 | 5.6 | 326.0 |
| 31.37 | 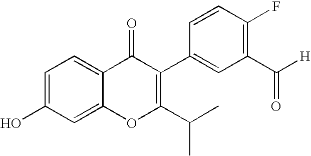 | | 327.3 |
| 31.38 | 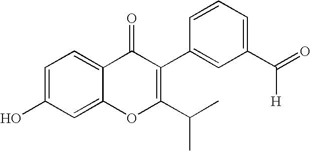 | | 309.3 |
| 31.39 | 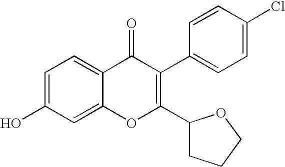 | 4.4 | 344.0 |
| 31.40 | 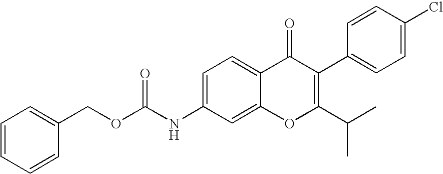 | 7.1 | 448.3 |
| 31.41 | 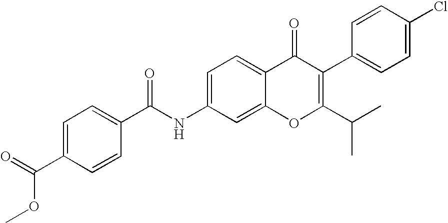 | 6.7 | 476.0 |
| 31.42 | 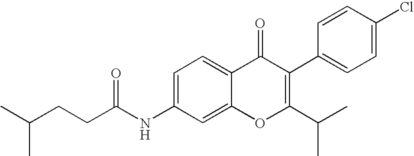 | 6.9 | 412.0 |
| 31.43 | 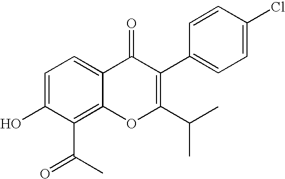 | | 357.8 |
| 31.44 | 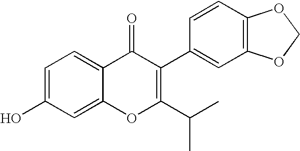 | | 325.0 |
| 31.45 | 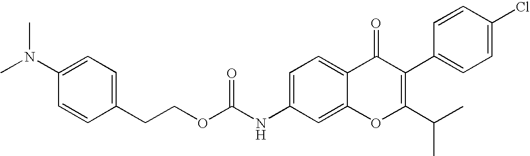 | 5.5 | 505.3 |
| 31.46 | 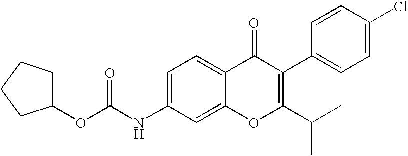 | 7.2 | 426.3 |
| 31.47 | 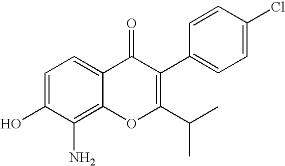 | 4.6 | 330.2 |
| 31.48 | 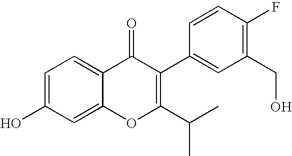 | | 329.3 |
| 31.49 | 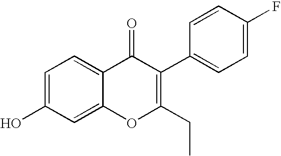 | 4.2 | 285.8 |
| 31.50 | 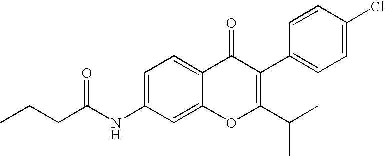 | 6.0 | 384.0 |
| 31.51 | 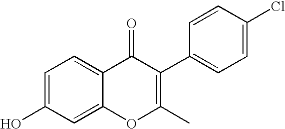 | 4.3 | 287.1 |
| 31.52 | 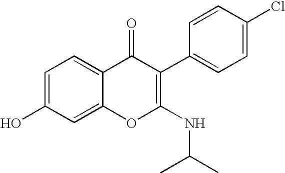 | 4.0 | 330.0 |
| 31.53 | 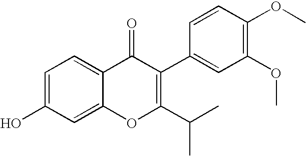 | | 341.4 |
| 31.54 | 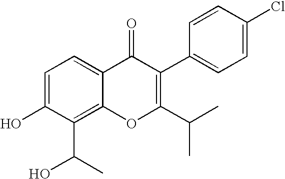 | 5.2 | 359.0 |
| 31.55 | 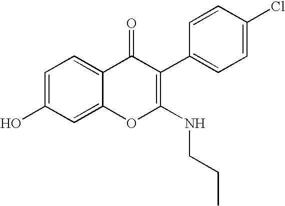 | 3.9 | 330.0 |
| 31.56 | 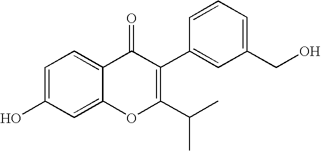 | 3.4 | 311.0 |
| 31.57 | 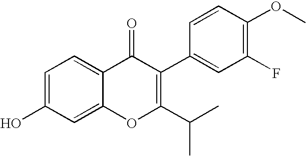 | | 329.3 |
| 31.58 | 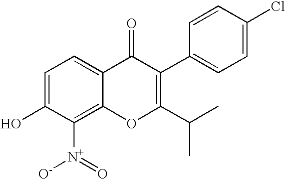 | 5.8 | 360.2 |
| 31.59 | 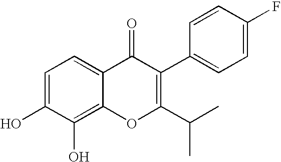 | 3.9 | 315.2 |
| 31.60 | 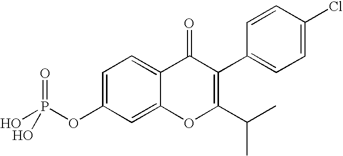 | 5.5 | 395.1 |
| 31.61 | 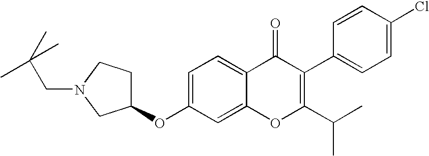 | | 455.4 |
| 31.62 | 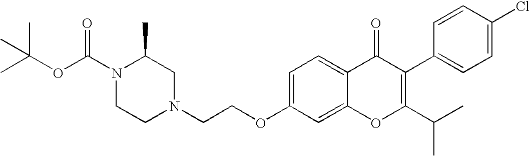 | | 542.1 |
| 31.63 | 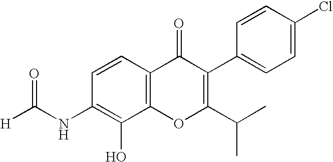 | 4.6 | 358.0 |
| 31.64 | 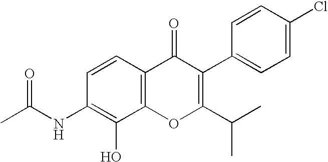 | 7.8 | 372.2 |
| 31.65 | 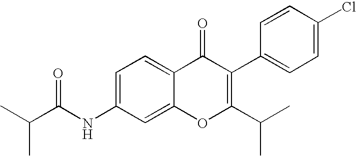 | 6.0 | 384.0 |
| 31.66 | 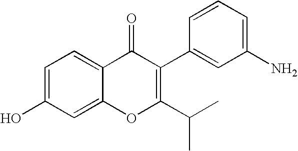 | 2.2 | 296.0 |
| 31.67 | 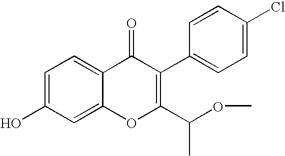 | 4.3 | 332.0 |
| 31.68 | 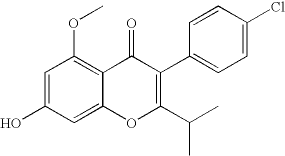 | 4.8 | 345.0 |
| 31.69 | 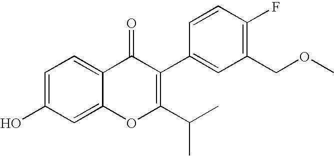 | | 343.4 |
| 31.70 | 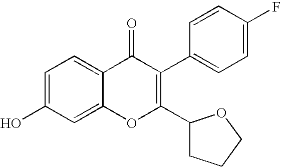 | 3.9 | 327.9 |
| 31.71 | 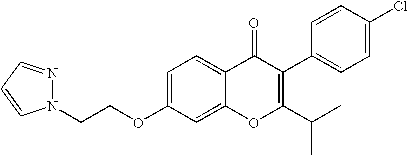 | 5.7 | 410.0 |
| 31.72 | 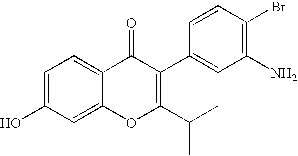 | 3.6 | 375.1 |
| 31.73 | 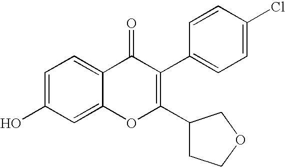 | 4.0 | 344.2 |
| 31.74 | 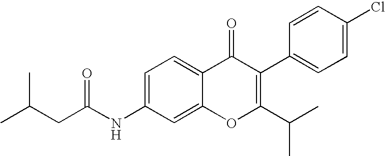 | 6.5 | 398.0 |
| 31.75 | 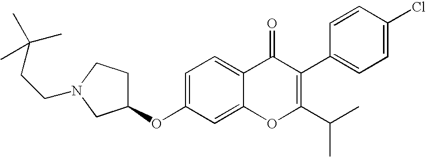 | | 469.4 |
| 31.76 | 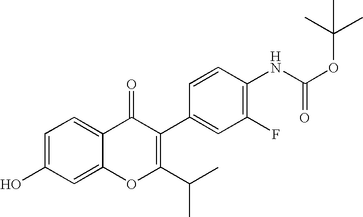 | 5.4 | 414.3 |
| 31.77 | 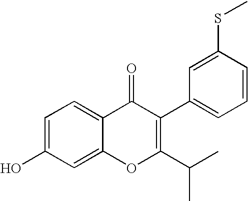 | | 327.4 |
| 31.78 | 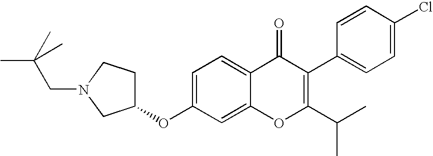 | | 455.4 |
| 31.79 | 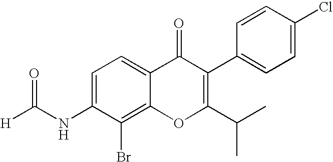 | 5.7 | 421.1 |

### EXAMPLE 32

### Preparation of Soft Gelatin Capsules

5,000 soft gelatin capsules, each comprising as active ingredient 0.05 g of one of the compounds of formula (Ia) mentioned in the preceding Examples, are prepared as follows:

### Composition

### Active Ingredient 250 g

### Lauroglycol® 2 l

The pulverized active ingredient is suspended in Lauroglykol® (propylene glycol laurate, Gattefosse S.A., Saint Priest, France) and ground in a wet pulverizer to produce a particle size of about 1-3 μm. 0.419 g portions of the mixture are then introduced into soft gelatin capsules using a capsule-filling machine.

1. A chromone compound of the formula 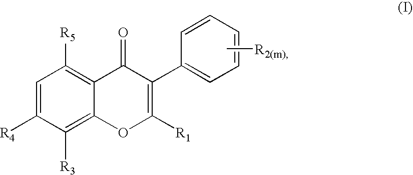
wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group 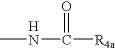
or a group 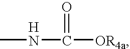
where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl;
R₅ is hydrogen, C₁-C₆alkoxy or hydroxy; and
m is 1, 2 or 3,
in free form or in salt form, and, where possible, in pharmaceutically acceptable acid addition salt form, for use as a pharmaceutical for the treatment or prevention of a disease or condition in which vanilloid receptor activation plays a role or is implicated.

2. The use of a chromone compound of formula (I): 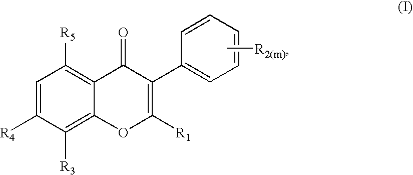
wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group 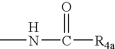
or a group 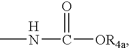
where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl;
R₅ is hydrogen, C₁-C₆alkoxy or hydroxy; and
m is 1, 2 or 3,
in free form or in salt form, and, where possible, in pharmaceutically acceptable acid addition salt form, for the manufacture of a medicament for the treatment or prevention of a disease or condition in which vanilloid receptor activation plays a role or is implicated.

3. A method for treating or preventing a disease or condition in which vanilloid receptor activation plays a role or is implicated comprising administering to a mammal in need thereof a therapeutically effective amount of a chromone compound of formula (I): 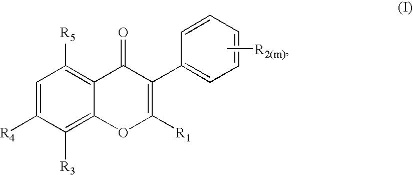
wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group 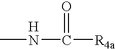
or a group 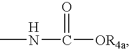
where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl;
R₅ is hydrogen, C₁-C₆alkoxy or hydroxy; and
m is 1, 2 or 3,
in free form or in salt form, and, where possible, in pharmaceutically acceptable acid addition salt form.

4. A chromone compound of formula 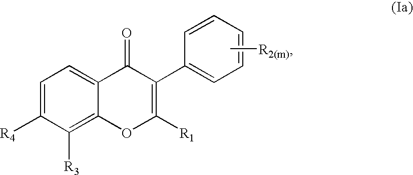
wherein
R₁ is C₁-C₆alkyl, (C₁-C₆alkyl)C₁-C₆alkyl, di-(C₁-C₆alkyl)C₁-C₆alkyl, C₃-C₆cycloalkyl, halogen, halogen-substituted C₁-C₆alkyl, (C₁-C₆alkoxy)C₁-C₆alkyl, tetrahydrofuryl or (C₁-C₆alkyl)amino;
each R₂, independently, is halogen, hydroxy, C₁-C₆alkoxy, C₁-C₆alkylthio, C₁-C₆alkyl, (C₁-C₆alkoxy)C₁-C₆alkyl, amino, C₁-C₆alkoxycarbonylamino, cyano, halogen-substituted C₁-C₆alkyl, hydroxyC₁-C₆alkyl or a group —C(═O)—R₂ₐ, where R₂ₐ is hydrogen or C₁-C₆alkyl, or, if m is 2 or 3, two radicals R₂ bound to adjacent carbon atoms can together also form a group —O—CH₂—O—;
R₃ is hydrogen, C₁-C₆alkyl, C₂-C₆alkenyl, amino, nitro, hydroxy, hydroxyC₁-C₆alkyl, halogen, C₁-C₆alkoxy, (C₃-C₆cycloalkyl)C₁-C₆alkoxy or a group —C(═O)—R₂ₐ, where R₂ₐ is hydrogen or C₁-C₆alkyl;
R₄ is hydroxy, esterified hydroxy, etherified hydroxy, amino, (C₁-C₆alkyl)amino, or a group 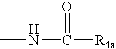
or a group 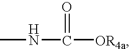
where R₄ₐ is hydrogen, C₁-C₆alkyl, (C₁-C₆alkoxycarbonyl)phenyl, benzyl, (C₁-C₆alkoxycarbonyl)benzyl, (C₁-C₆alkoxycarbonyl)piperidyl, (di-(C₁-C₆alkyl)amino)phenethyl or C₃-C₆cycloalkyl; and
m is 1, 2 or 3,
in free form or in salt form, and, where possible, in acid addition salt form, with the proviso, that, when R₂ is halo, m is 1, R₃ is hydrogen or hydroxy and R₄ is hydroxy, then R₁ is other than methyl.

5. A pharmaceutical composition comprising a compound of claim 4 in free or salt form and, where possible, in pharmaceutically acceptable acid addition salt form, in association with a pharmaceutical carrier or diluent.

6. A process for the preparation of a compound of formula (Ia), as defined in claim 4, or a salt thereof, comprising:
a) for the preparation of a compound of formula (Ia), where R₁ is as defined in claim 4, R₂ is chloro, R₃ is hydrogen, R₄ is hydroxy and m is 1, reacting resorcinol with 4-chlorophenylacetic acid in the presence of boron trifluoride etherate in a first step to obtain the ethanone compound having the formula 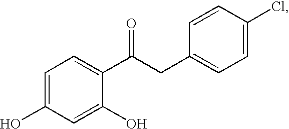
which compound is then reacted with an anhydride of the formula
O—(COR₁)₂,
in the presence of an organic base to obtain an ester compound having the formula 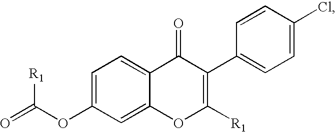
which compound is then hydrolysed with aqueous potassium hydroxide to obtain a chromen-4-one compound having the formula 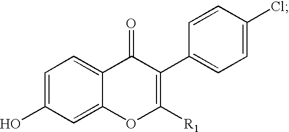
b) for the preparation of a compound of formula (Ia), where R₁ is as defined in claim 4, R₂ is chloro, R₃ is methoxy, R₄ is hydroxy and m is 1, reacting the chromen-4-one compound prepared in a) above with hexamethylenetetramine in the presence of acetic acid to obtain an imine compound which is then reacted with hydrochloric acid to obtain a carbaldehyde compound having the formula 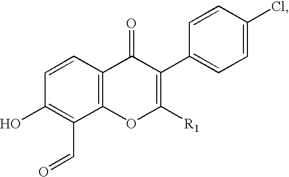
which compound is then reacted with benzyl bromide to obtain a benzylated carbaldehyde compound having the formula 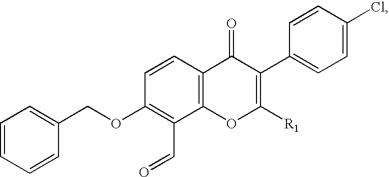
which compound is then oxidised with m-chloroperbenzoic acid and then treated with an aqueous potassium hydroxide solution to obtain a chromen-4-one compound having the formula 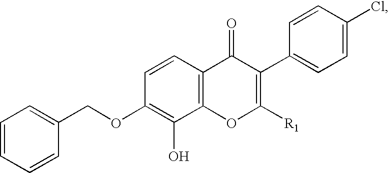
which compound is then alkylated with iodomethane in the presence of potassium carbonate to obtain a chromen-4-one compound having the formula 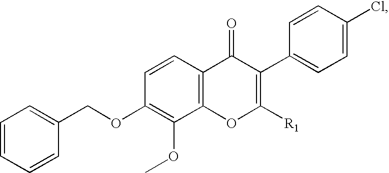
which compound is then debenzylated with palladium on carbon to obtain a chromen-4-one compound having the formula 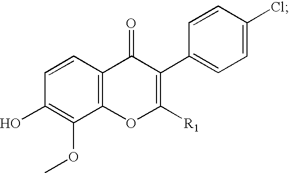
c) for the preparation of a compound of formula (Ia), where R₁ is as defined in claim 4, R₂ is chloro, R₃ is C₂-C₆alkyl, R₄ is hydroxy and m is 1, reacting a carbaldehyde compound having the formula 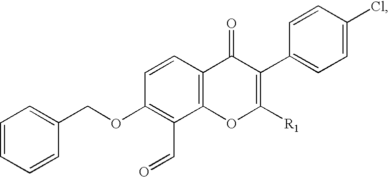
with a mixture of sodium hydride and an alkyl triphenylphosphonium bromide to obtain an 8-alkenyl substituted chromen-4-one compound having the formula 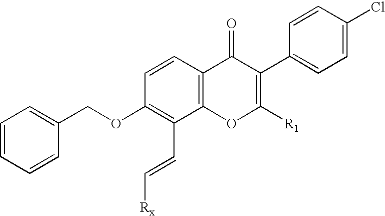
where Rₓ is hydrogen or C₁-C₄alkyl, which compound is then debenzylated/hydrogenated with palladium on carbon to obtain an 8-alkyl substituted chromen-4-one compound having the formula 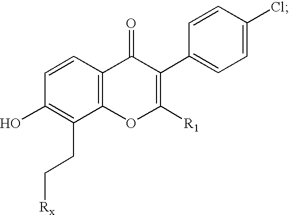
d) for the preparation of a compound of formula (Ia), where R₁, R₂ and m are as defined in claim 4, R₃ is hydrogen and R₄ is hydroxy, reacting 2,4-dihydroxyacetophenone with 4-methoxybenzyl chloride to obtain the ethanone compound having the formula 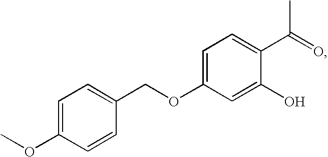
which compound is then acylated with an alkanoyl chloride having the formula R₁COCl to obtain an ester compound having the formula 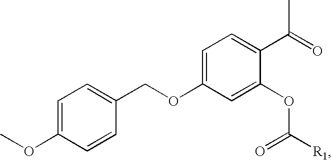
which compound is then reacted with sodium hydride and then treated with aqueous ammonium hydroxide to obtain a compound having the formula 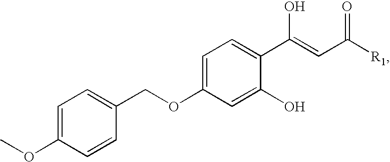
which compound is then reacted with t-butyldimethylsilylchloride to obtain the silylised compound having the formula 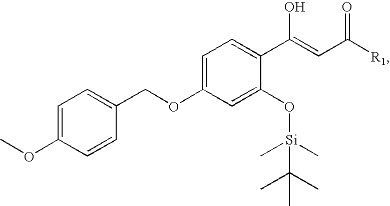
which compound is then reacted with N-bromosuccinimide to obtain a dione compound having the formula 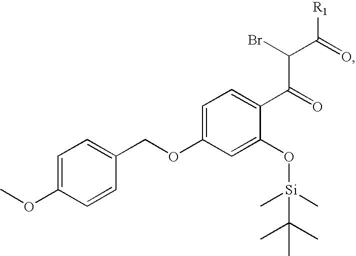
which compound is then desilylated/cyclised/debenzylated by reacting it with concentrated sulphuric acid to obtain a 3-bromo-substituted chromen-4-one compound having the formula 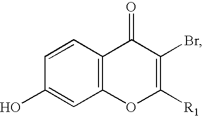
which compound is then reacted with a phenyl substituted boronic acid having the formula 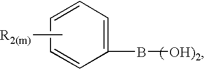
to obtain a chromen-4-one compound having the formula 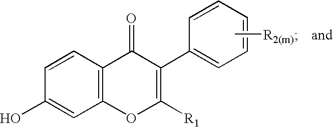
e) for the preparation of a compound of formula (Ia), where R₁ is as defined in claim 4, R₂ is chloro, R₃ is hydrogen, R₄ is amino, (C₁-C₆alkyl)amino, a group 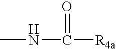
or a group 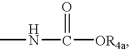
where R₄ₐ is as defined in claim 4, and m is 1, reacting the chromen-4-one compound prepared in a) above with triflic anhydride to obtain a trifluoromethane sulfonic ester compound having the formula 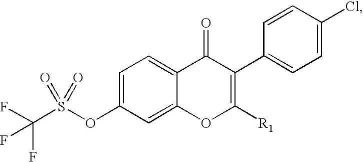
which compound is then reacted with benzophenone imine having the formula 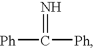
where R₁ is as defined above, to obtain a 7-benzhydrylidene-substituted chromen-4-one having the formula 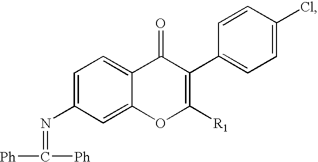
which compound is then subjected to acid hydrolysis to obtain a chromen-4-one compound having the formula 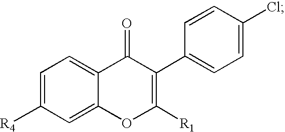
wherein R₄ is NH₂;
and optionally subjecting the obtained chromen-4-one compound to reductive alkylation utilising an aldehyde or ketone, a reaction with a C₁-C₆alkyl halide, an acylation reaction with an acyl chloride of the formula 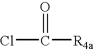
or a reaction a compound with an alkylchloroformate of the formula 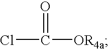
where R₄ₐ in both cases is as defined in claim 4
and recovering the corresponding compounds prepared in a)-e) in free or salt form.
